SEQUENCE LISTING (15 sequences)

SEQ ID NO: 1 - protein, 7 aa, Artificial Sequence
  Description of Artificial Sequence Synthetic peptide
  MOD_RES (7)..(7): Dehydroalanine
VYRFYGA

SEQ ID NO: 2 - protein, 7 aa, Artificial Sequence
  Description of Artificial Sequence Synthetic peptide
VWRFHGG

SEQ ID NO: 3 - protein, 7 aa, Artificial Sequence
  Description of Artificial Sequence Synthetic peptide
VQRYWGG

SEQ ID NO: 4 - protein, 7 aa, Artificial Sequence
  Description of Artificial Sequence Synthetic peptide
VYRFYGG

SEQ ID NO: 5 - protein, 7 aa, Artificial Sequence
  Description of Artificial Sequence Synthetic peptide
VLQWFGG

SEQ ID NO: 6 - protein, 7 aa, Artificial Sequence
  Description of Artificial Sequence Synthetic peptide
VRLWFGG

SEQ ID NO: 7 - protein, 7 aa, Artificial Sequence
  Description of Artificial Sequence Synthetic peptide
VILTFGG

SEQ ID NO: 8 - protein, 7 aa, Artificial Sequence
  Description of Artificial Sequence Synthetic peptide
VRLMFGG

SEQ ID NO: 9 - protein, 7 aa, Artificial Sequence
  Description of Artificial Sequence Synthetic peptide
  MOD_RES (7)..(7): Dehydroalanine
VWRFHGA

SEQ ID NO: 10 - protein, 7 aa, Artificial Sequence
  Description of Artificial Sequence Synthetic peptide
  MOD_RES (7)..(7): Dehydroalanine
VQRYWGA

SEQ ID NO: 11 - protein, 7 aa, Artificial Sequence
  Description of Artificial Sequence Synthetic peptide
  MOD_RES (7)..(7): Dehydroalanine
VLQWFGA

SEQ ID NO: 12 - protein, 7 aa, Artificial Sequence
  Description of Artificial Sequence Synthetic peptide
  MOD_RES (7)..(7): Dehydroalanine
VRLWFGA

SEQ ID NO: 13 - protein, 7 aa, Artificial Sequence
  Description of Artificial Sequence Synthetic peptide
  MOD_RES (7)..(7): Dehydroalanine
VILTFGA

SEQ ID NO: 14 - protein, 7 aa, Artificial Sequence
  Description of Artificial Sequence Synthetic peptide
  MOD_RES (7)..(7): Dehydroalanine
VRLMFGA

SEQ ID NO: 15 - protein, 6 aa, Artificial Sequence
  Description of Artificial Sequence Synthetic 6xHis tag
HHHHHH

(19) United States
(12) Patent Application Publication
(10) Pub. No.: US 2017/0327549 A1
(43) Pub. Date: Nov. 16, 2017

(54) Adenylation Enzyme Inhibitors

(71) Applicant: Stichting Het Nederlands Kanker Instituut - antoni van Leeuwenhoek Ziekenhuis, Amsterdam (NL)

(72) Inventors: Huib Ovaa, Amsterdam (NL); Farid El Oualid, Amsterdam (NL); Monique Mulder, Amsterdam (NL)

(73) Assignee: Stichting Het Nederlands Kanker Instituut - Antoni van Leeuwenhoek Ziekenhuis, Amsterdam (NL)

(21) Appl. No.: 15/506,624

(22) PCT Filed: Aug. 26, 2015
(86) PCT No.: PCT/NL2015/050596

§ 371 Date: Feb. 24, 2017

(30) Foreign Application Priority Data
Aug. 26, 2014 (NL) 2013366

(51) Int. Cl. C07K 14/47; G01N 33/58; C12Q 1/25; C12Q 1/48; A61K 38/00

(52) U.S. Cl. CPC C07K 14/4702; C12Y 603/02019; C12Q 1/25; C12Q 1/48; C12Y 203/02; G01N 33/581; A61K 38/00; G01N 2333/9015; G01N 2333/9108; G01N 2440/40

(57) ABSTRACT

The present invention concerns compounds that are capable of covalently entrapping adenylating enzymes. The present invention is essentially based on the discovery that analogues of adenylating enzyme (AE) substrates, wherein a methylene group has been incorporated at the carbon atom in the α-position relative to the carboxylate group involved in the adenylation, are capable of undergoing the adenylation reaction, thereby creating an activated methylene group in situ. The resulting ‘armed’ acyladenylate can interact with the enzyme resulting in covalent entrapment. Interestingly, the acyladenylate can alternatively be transferred to the next step in the enzyme cascade, following which the activated methylene group can interact with the next (active site cysteine containing) enzyme in the enzymatic cascade. The AE substrate analogues, based on their capability of ‘entrapping’ the respective enzymes, will have utility as activity based probes in biological research and also as diagnostic and/or therapeutic agents.

### FIELD OF THE INVENTION

The present invention concerns compounds that are capable of covalently entrapping adenylating enzymes. The invention also concerns methods of preparing such compounds and the use thereof as activity based probe, as diagnostic agent and as therapeutic agent.

### BACKGROUND OF THE INVENTION

Adenylation is a biological process used to chemically activate carboxylate substrates by condensing them with ATP to liberate pyrophosphate (the driving force). The mechanism proceeds through a negatively pentavalent phosphorus atom which must be stabilized. Adenylate-forming enzymes catalyze the difficult condensation between the weakly nucleophilic carboxylic acid and weakly electrophilic phosphate. Binding of a carboxylic acid substrate and ATP is followed by nucleophilic attack of the substrate carboxylate on the α-phosphate of ATP to generate acyladenylate and the release of pyrophosphate. In a second reaction, the enzyme binds an acceptor molecule and transfers the acyl group of the acyladenylate onto a nucleophilic amino, alcohol, or thiol group of an acceptor molecule to generate the desired amide, ester, or thioester products, respectively, thereby liberating AMP. Necessarily it seems that the enzymes that make the intermediate also catalyze the second step. The process of adenylation occurs in a variety of metabolic pathways. Adenylating enzymes have been identified that catalyze essential biochemical processes in protein synthesis and metabolism, glycolysis, lipid metabolism, and cofactor biosynthesis (biotin, coenzyme A, and nicotine adenine dinucleotide) as well as synthesis of small molecule metabolites including the mycobactins (siderophores for iron acquisition) and mycothiols (a thiol to protect against oxidative stress).

Not surprisingly, considerable effort has been put into the development of analogues capable of covalently modifying the adenylating enzyme active sites. Such analogues will have utility in biological research (activity-based probe or ABP), as diagnostic agents and/or as therapeutic agents.

One of the most straightforward strategies to prepare such analogues involves the design of a mimic of the acyladenylate. The rationale is based on the findings that the intermediate acyladenylates bind several orders of magnitude more tightly than the substrate acids since they simultaneously occupy both substrate-binding pockets (acid and ATP). Thus acyladenylate analogues that incorporate a stable bioisostere of the labile acylphosphate linkage function can potentially provide potent bisubstrate inhibitors. Isosteric replacements of the native acylphosphate linkage of the acyladenylate that have been investigated include alkyl phosphates, β-ketophosphonates, acylsulfamates and sulfamates. In particular acyl sulfonyladenosines have been investigated extenisvely, since this most closely resembles the overall molecular geometry and charge distribution of the acylphosphate. Inspired by sulfamoyladenosine natural products such as nucleocidin and ascamycin, acyl sulfonyladenosines were used to produce inhibitors of various enzymes in this mechanistic superfamily, including aminoacyl-tRNA synthetase, members of the ANL family, E1 activating enzymes, asparagine synthetase, and pantothenate synthetase. Bisubstrate inhibitors incorporating the sulfonyladenosine structure typically possess potent nanomolar dissociation constants and display good biochemical selectivity towards other adenylating enzymes.

Adenylation often is just a step in a more complex enzymatic cascade. For example, in the ubiquitin (and ubiquitin like) conjugation pathway(s), after initial ATP-dependent activation by an E1 ubiquitin-activating enzyme (E1), the C-terminal carboxyl group of ubiquitin forms a high-energy thioester bond with an active Cys group of the E1 enzyme, followed by the activated ubiquitin being transferred to a specific Cys residue of one of a family of E2 ubiquitin-conjugating enzymes (E2s) via a similar thioester linkage. The E3 ubiquitin ligases (E3s) play a critical role in the ubiquitin conjugation cascade by recruiting ubiquitin-loaded E2s, recognizing specific substrates, and facilitating or directly catalyzing ubiquitin transfer to either the Lys residues (in most cases) or the N terminus of their molecular targets.

Currently existing acyladenylate analogues, such as the ones described above, typically have been designed primarily with the aim of entrapping the adenylating enzyme; they usually lack the capability of being transferred to enzymes involved in reactions downstream the cascade. It would be highly advantageous to have available analogues that have the capability to be transferred downstream so that they can target various or all parts of the enzyme cascade. Analogues having such capabilities would be particularly valuable tools in biological research and potentially in diagnostic and/or therapeutic applications.

It would also be highly advantageous to have a strategy for the design of analogues based on the use of a simple building block which can be incorporated using relatively straight forward synthetic procedures, which would open up the way to the design and synthesis of a wide range of analogues.

It is the aim of the present invention to provide a novel class of such analogues that have such capabilities.

### SUMMARY OF THE INVENTION

The present inventors surprisingly discovered that analogues of adenylating enzyme (AE) substrates, wherein a methylene group has been incorporated at the carbon atom in the α-position relative to the carboxylate group involved in the adenylation, are capable of undergoing the adenylation reaction, thereby creating an activated methylene group in situ. The resulting ‘armed’ acyladenylate can interact with the enzyme in two distinct ways: the activated methylene group can react with the cysteine residue in the active site of the enzyme resulting in covalent entrapment; or the acyladenylate follows the normal route to be transferred to the next step in the enzyme cascade, during which the activated methylene group can again interact with the (active site cysteine containing) enzyme in these two distinct manners (see also FIG. 1a).

This concept is demonstrated in the experimental part, where the development of the ubiquitin analogue Ub(1-75)-ΔAla (ΔAla=dehydroalanine) is described, which analogue was shown to be capable of “travelling” through the entire E1-E2-E3 machinery. More in particular, a probe for the full conjugation machinery was developed that contains the native Ub structure required for processing by the E1-E2-E3 machinery that bears an electrophilic trap that does not necessarily hamper transfer to an E2 and E3. First, Ub(1-75)-ΔAla is converted with ATP by E1 into an adenylate. Next, there are two possible pathways: 1) the E1 active site cysteine residue reacts with the activated methylene group of the ΔAla moiety, inactivating the E1 or 2) the E1 active site cysteine residue follows the native route and attacks the carbonyl of the AMP ester. The probe is then charged onto the E1 as an active-site thioester and because of its native character this can be transferred to an E2. During this transfer the same two possibilities exist: the E2 active site cysteine forms a covalent adduct with the probe or forms a native Ub-E2 thioester intermediate. Finally, the same applies for the E2 to E3 transfer (only HECT and RBR type E3), enabling the Ub analogue (Ub(1-75)-ΔAla) to “travel” through the E1-E2-E3 machinery. The Ub(1-75)-ΔAla structure allows covalent labeling of each of the E1-E2-E3 enzyme classes of the Ub/Ub1 conjugation machinery, which is possible by the dual role of the ΔAla moiety as a Gly76 mimic and an in-situ generated electrophile. Interestingly, it was also established by the present inventors, as described in the experimental part, that by certain manipulations to the conditions under which the analogue and the enzyme(s) are incubated, the reaction can be directed into one of the two possible ways (i.e. covalent binding through the methylene group or the ‘native’ reaction pathway). For example, it was established that trapping of E1 by Ub(1-75)-ΔAla was most effective at a pH value of approximately 8, while trapping of E2 enzymes by Ub(1-75)-ΔAla was most effective at a pH value of around 7.5.

The concept of the present invention has also been demonstrated with the ΔAla analogues of Nedd8, SUMO1, SUMO2 and SUMO3, as described in more detail in the experimental part.

Hence, it is apparent to those of ordinary skill in the art that the strategy described herein is generally applicable, meaning that it can be used to produce analogues of AE substrates of a diverse (biological) nature, including proteins, peptides, carbohydrates, fatty acids and organic small molecule compounds.

The present strategy is typically based on the introduction in the AE substrate of a dehydroalanine (ΔAla) moiety or a comparable acrylic acid moiety. There are various methods for the synthesis of the ΔAla group. For example, (alkylated) cysteine can be used as a building block in the synthesis of the analogue, which can be transformed into ΔAla by oxidative elimination. Strategies based on the incorporation of ΔAla are generally applicable, i.e. in the synthesis of peptide as well as non-peptide analogues.

As will be understood by those of average skill in the art, the AE substrate analogues of this invention, based on their capability of ‘entrapping’ the respective AEs, will have utility as activity based probes in biological research and also potentially as diagnostic and/or therapeutic agents.

The present invention concerns the AE substrate analogues. The invention also concerns methods of synthesizing and modifying selected AE substrates using the present strategy to obtain analogues. Furthermore, the invention concerns the intermediate and endproducts obtained in such methods. Furthermore, the invention concerns the use of these analogues as activity based probes and/or as AE inhibitors in diagnostic and/or therapeutic methods. These and other aspects of the invention, as defined in the appending claims, will be described and exemplified in more detail in the following description and examples.

### DETAILED DESCRIPTION OF THE INVENTION

Hence, in a first aspect, the present invention provides AE substrate analogues having the structure:

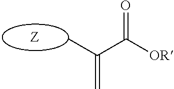

wherein R′ represents hydrogen or a moiety represented by the formula

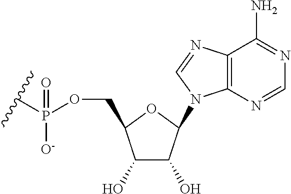 [SMILES: *P(=O)([O-])OC[C@H]1O[C@@H](n2cnc3c(N)ncnc32)[C@H](O)[C@@H]1O]

preferably R′ represent hydrogen; and wherein

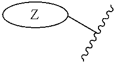

represents an organic moiety selected from the group consisting of peptides, hydrocarbons, carbohydrates and low-molecular weight organic moieties; said compound having the capability of binding to an adenylation enzyme (AE) and/or to act as an inhibitor of an adenylating enzyme.

In a particularly preferred embodiment of the invention

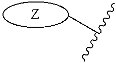

represents an organic moiety selected from the group consisting of peptides, hydrocarbons, carbohydrates and low-molecular weight organic moieties, said organic moiety being identical to or resembling the corresponding part of a C-terminal carboxylate containing AE substrate. It will be understood by those skilled in the art what is meant with the term ‘corresponding part’, given the principle that the compounds of the present invention, in essence, are modified versions of natural AE substrates, said AE substrates having a carboxylic acid moiety that is involved in the adenylation reaction by the enzyme, said modification being the ‘introduction’ of a methylene group at the α carbon atom. As will be understood by those skilled in the art, the atoms in the main chain of the AE substrate are denominated relative to the carboxylic acid moiety that is involved in the adenylation, the carbon atom adjacent to the carboxylate moiety being designated a. The ‘corresponding part’ therefore refers to the natural AE substrate minus the carboxylic acid group and the moiety in the α position (e.g. the —CH₂— group in the α position). Furthermore, it will also be understood by those skilled in the art, that certain further modifications to the AE substrate are envisaged, in particular to the atom in the β position.

The synthesis of the AE substrate analogue, in one preferred embodiment of the invention, involves the introduction of an dehydroalanine moiety that ‘replaces’ the carboxyl group as well as the ‘moieties’ in the α and β positions.

In a preferred embodiment of the invention, an AE substrate analogue as defined here above, is provided having the structure:

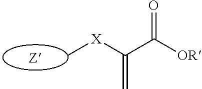

wherein R′ has the same meaning as defined here above, X represents —NH—, —O— or —CH₂— preferably —NH—; and

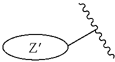

represents an organic moiety selected from the group consisting of peptides, hydrocarbons, carbohydrates and low-molecular weight organic moieties; preferably an organic moiety selected from the group consisting of peptides, hydrocarbons, carbohydrates and low-molecular weight organic moieties, said organic moiety being identical to or resembling the corresponding part of a C-terminal carboxylate containing AE substrate said AE substrate analogue having the capability of being recognized and binding to an adenylation enzyme (AE) and/or to act as an inhibitor of the adenylating enzyme. As will be understood by those skilled in the art, based on the explanation above, the ‘corresponding part’ in this embodiment refers to the natural AE substrate minus the carboxylic acid group, the moiety in the α position (e.g. the —CH₂— group in the α position) and the moiety in the β position (e.g. the —CH₂— group in the β position).

As will be understood by those skilled in the art, based on the explanation herein and on the experimental data provided in the examples, the proposed modification(s) of the AE substrate to produce the analogue typically do not significantly hamper the substrates capability of being recognized by and interacting with the adenylating enzyme. Hence, the compounds as defined above are characterized by the capability of being recognized by and binding to the respective adenylating enzyme. By virtue of the proposed modification(s) the compounds defined above in addition have the capability of acting as an inhibitor of the respective adenylating enzyme and therefore have potential utility as activity based probes, e.g. in biological research, and/or as diagnostic and/or therapeutic agent.

The ability of a compound of the invention to act as an inhibitor of an adenylating enzyme can be determined using pharmacological models which are well known to the person skilled in the art, or using any of the assays described below.

For example, the inhibitory activity of the compounds may be determined using, in particular, a suitable competition binding assay with adenylating enzymes. Typically, in accordance with the invention, an IC₅₀ value of the compound, as determined by these assays, of less than 10 μM, preferably less than 1 μM is indicative of the capability of a compound to act as an inhibitor of an adenylating enzymes. In some embodiments of the invention, the compounds have an IC₅₀ value, as determined by these assays of less than 500 nM, more preferably less than 250 nM, more preferably less than 100 nM, more preferably less than 50 nM, less than 25 nM, less than 15 nM, less than 10 nM, less than 5 nM, or less than 1 nM.

An ATP-[³²P]PPᵢ exchange assay can also be used to further characterize the AE substrate analogues of this invention. The assay exploits the equilibrium nature of the acylation reaction which can be summarized as follows: E+S+ATP=>[E−S−AMP]+PPᵢ. Measurement in the reverse direction allows one to examine substrate selectivity in a steady-state process. Thus, ³²PPᵢ is added and its incorporation to [³²P]ATP is measured. The ³²P-ATP formed is isolated by adsorption onto charcoal and the charcoal is washed to remove [³²P]PPᵢ. The charcoal is transferred to a scintillation vial and counts are measured using liquid-scintillation-counting. The counts obtained from above are used to determine the initial velocity of ³²P-ATP formation (ν₀). Measuring the initial velocity in the presence of various concentrations of inhibitor allows one to measure the Kᵢ values of the inhibitors. Such a method has, for example, been used by Zhao et al. (‘Inhibiting the Protein Ubiquitination Cascade by Ubiquitin-Mimicking Short Peptides’, Organic Letters, 2012 Vol. 14, 5760) and by Zhao et al. (Phage display to identify Nedd8-mimicking peptides as inhibitors of the Nedd8 transfer cascade’, ChemBiochem 2013, Vol 14, 1323). The contents of which, in particular with regard to the determination of kinetic parameters, are incorporated herein by reference.

Typically, in accordance with the invention, a Kᵢ value of the compound, as determined by an ATP-PPi exchange assay, at or below the concentration of the enzyme used in the assay is indicative of AE substrate analogue's capability of being recognized and binding to an adenylation enzyme (AE) and/or to act as an inhibitor of the adenylating enzyme.

The ability of a compound of the invention to act as an inhibitor of an adenylating enzyme can also be determined in a more high-throughput fashion using recently developed methods: one is based on using fluorogenic ATP analogues that undergo a large change in fluorescence characteristics upon enzymatic processing (Hacker et al., Angewandte Chemie Int. Ed. 2013, Vol 52, 11916); the second is a colorimetric assay that allows spectrophotometrically monitoring the amount of released pyrophosphate (PPᵢ) by breaking it down with pyrophosphatase into phosphate, which is combined with molybdate to form the blue phosphomolybdate complex (Berndsen and Wolberger, Analytical Biochemistry 2011, Vol 418, 102).

The ability of a compound of the invention to act as an inhibitor of an adenylating enzyme can also be determined in a coupled continuous spectrophotometric assay that employs hydroxylamine as a surrogate acceptor molecule leading to the formation of a hydroxamate. The released pyrophosphate from the first half-reaction is measured using the pyrophosphatase-purine nucleoside phosphorylase coupling system with the chromogenic substrate 7-methylthioguanosine (Anal Biochem. 2010 Sep. 1; 404(1): 56-63).

The ability of a compound of the invention to act as an inhibitor of an adenylating enzyme can also be determined using the AMP-Glo™ Assay (by Promega). The AMP-Glo™ Assay(a) is a homogenous assay that generates light signal from any reaction that produces AMP as a reaction product. The assay is designed to quantitatively monitor the concentration of AMP in a biochemical reaction in a high-throughput format (U.S. Pat. No. 6,599,711 and U.S. Pat. No. 6,911,319).

In one embodiment, the present invention concerns AE substrate analogues, in the form of peptides or proteins, characterized in that said peptides or proteins are identical to or resemble a natural or ‘wild-type’ AE substrate protein or peptide modified by the substitution of the C-terminal amino acid residue by a dehydroalanine residue. The invention however also entails the modification of non-natural peptides and proteins comprising an amino acid sequence that is recognized by an adenylating enzyme. Many examples of such non-natural peptides that have the capability of being recognized by and interact with an adenylating enzymes have been described in the art. For illustrative purposes Zhao et al. (‘Inhibiting the Protein Ubiquitination Cascade by Ubiquitin-Mimicking Short Peptides’, Organic Letters, 2012 Vol. 14, 5760) and by Zhao et al. (Phage display to identify Nedd8-mimicking peptides as inhibitors of the Nedd8 transfer cascade’, ChemBiochem 2013, Vol 14, 1323) may be referred, the contents of which, in particular with regard to the identity and structure of the Ubiquitin and Nedd8-mimicking peptides, are incorporated herein by reference.

The natural or ‘wild-type’ AE substrate peptides and proteins and the non-natural peptides and proteins comprising an amino acid sequence that is recognized by an adenylating enzyme, that may be modified in accordance with the invention to provide AE targeting ABP's or inhibitors, are collectively referred to herein as ‘reference AE substrate’.

As will be understood by those skilled in the art, a protein or peptide that is a substrate or target for an adenylating enzyme contains a specific sequence of amino acids that results in recognition by and interaction with the adenylating enzyme. Said sequence is referred to herein as ‘recognition sequence’. The recognition site for an adenylating enzyme will always include the C-terminal part of protein or peptide sequence, the terminal carboxyl group thereof being the target for adenylation.

For ease of reference, the amino acid residues in the protease substrate and, hence, the corresponding AE substrate analogue, are thus identified herein based on their position in the protein backbone relative to the C-terminal amino acid residue. In the context of the present invention the amino acid positions are designated 1, 2, 3, . . . , ω, wherein 1 denotes the C-terminal amino acid position and ω denotes the N-terminal amino acid position. The amino acids at these positions are designated P¹, P², P³, . . . , P^{ω}), wherein P¹ is thus used to denote the amino acid containing the terminal carboxyl group.

Hence, in a particularly preferred embodiment of the invention, an AE substrate analogue peptide or protein is provided having the structure according to formula (I):

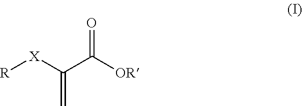

wherein R′ represents hydrogen or a moiety represented by the formula:

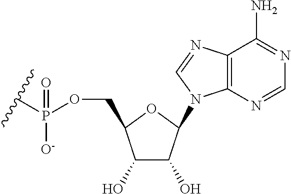 [SMILES: *P(=O)([O-])OC[C@H]1O[C@@H](n2cnc3c(N)ncnc32)[C@H](O)[C@@H]1O]

preferably R′ represent hydrogen; and wherein: X represents —NH— or —O—, preferably —NH—; and R represents an amino acid or a peptide comprising two or more amino acid residues; preferably R represents
a peptide having the structure of formula (ii)

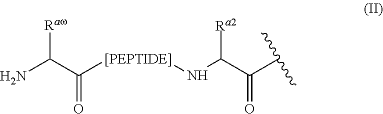

wherein R^{a#} represents an amino acid side chain identical to the amino acid side chain of the amino acid at the corresponding position in a reference AE substrate peptide; and [PEPTIDE] represents a peptide chain having the amino acid sequence —[P^{ω-1}-P³]— wherein P^{#} represents an amino acid residue identical to the amino acid residue in the corresponding position in said reference AE substrate peptide, wherein the positions are defined relative to C-terminal amino acid position, P¹ representing the C-terminal amino acid residue and P^{ω} representing the N-terminal amino acid residue;
an N-terminally truncated variant of said peptide having the structure of formula (ii), said N-terminally truncated variant comprising a number of amino acid residues of equal to or higher than 1, preferably equal to or higher than 2, more preferably equal to or higher than 5; or
a homologue of said peptide or N-terminally truncated variant thereof.

As mentioned above, R^{a} represents an amino acid side chain, which may be the side chain of a natural amino acids, an unnatural amino acid or a beta-amino acid. In one embodiment it is the amino acid side chain of one of the proteinogenic amino acids, i.e. a side chain of an amino acid selected from the group consisting of Histidine; Alanine; Isoleucine; Arginine; Leucine; Asparagine; Lysine; Aspartic acid; Methionine; Cysteine; Phenylalanine; Glutamic acid; Threonine; Glutamine; Tryptophan; Glycine; Valine; Proline; Selenocysteine; Serine; and Tyrosine.

The preferred meaning depends on the AE substrate analogue to be produced with the building block. In particular, it will be preferred, in one embodiment, that R^{a} corresponds to the amino acid side chain of the amino acid residue at the corresponding position in the reference AE substrate. Hence, the invention is not particularly limited in this regard.

As will be clear from the explanation and definitions above [PEPTIDE], in formula (ii), typically represents an amino acid sequence, identical to the corresponding portion of the reference AE substrate. In this context ‘corresponding portion’ means the amino acid sequence found in the reference AE substrate at the same position relative to the cleavage site. For example, if the adenylating enzyme is an E1 enzyme from the Ubiquitin conjugation pathway, the reference AE substrate is ubiquitin and the amino acid chain p^{ω}-p¹ represents the entire naturally occurring ubiquitin sequence and [PEPTIDE] in formula (I) thus typically defines said entire ubiquitin sequence minus the N-terminal amino acid ad minus the two C-terminal amino acids.

As explained herein before, the present strategy can be used to produce an analogue of any adenylating an enzyme that is capable of targeting also enzymes downstream in the cascade. In one particularly preferred embodiment of the invention, the E1-E2-E3 machinery in the ubiquitin and ubiquitin-like conjugation pathways is targeted.

The term ‘Ubiquitin-like protein’ (UBL) is a term of the art. UBLs modify cellular targets in a pathway that is parallel to, but distinct from, that of ubiquitin. UBLs are structurally similar to ubiquitin and are processed, activated, conjugated, and released from conjugates by enzymatic steps that are similar to the corresponding mechanisms for ubiquitin. UBLs are also translated with C-terminal extensions that are processed to expose the invariant C-terminal Gly residue. These modifiers have their own specific E1 (activating), E2 (conjugating) and E3 (ligating) enzymes that conjugate the UBLs to intracellular targets. These conjugates can be reversed by UBL-specific isopeptidases that have similar mechanisms to that of the deubiquitinating enzymes

Ubiquitin-like conjugation pathways include the SUMO conjugation pathway, the NEDD8 conjugation pathway, the ISG15 conjugation pathway, the FAT10 conjugation pathway, the MNSFβ conjugation pathway, the UFM1 conjugation pathway, the ATG8 conjugation pathway, the ATG12 conjugation pathway, the APG8 conjugation pathway, and the URM1 conjugation pathway. As will be understood by those skilled in the art, the adenylating enzyme involved in each of these pathways is the E1 enzyme. Hence in a preferred embodiment of the invention the adenylating enzyme is an E1 enzyme involved in the ubiquitin conjugation pathway, such as Uba1 and Uba6, an E1 enzyme involved in the SUMO conjugation pathway, such as SAE1/Uba2, an E1 enzyme involved in the NEDD8 conjugation pathway, such as NAE1/Uba3, an E1 enzyme involved in the ISG15 pathway, such as Uba7, an E1 enzyme involved in the FAT10 conjugation pathway, an E1 enzyme involved in the MNSFβ conjugation pathway, an E1 enzyme involved in the UFM1 conjugation pathway, an E1 enzyme involved in the ATG8 conjugation pathway, an E1 enzyme involved in the ATG12 conjugation pathway, an E1 enzyme involved in the APG8 conjugation pathway or an E1 enzyme involved in the URM1 conjugation pathway.

Hence, in an embodiment of the invention an AE substrate analogue as defined herein is provided, wherein the adenylating enzyme is selected from the aforementioned groups of enzymes.

Accordingly, in an embodiment of the invention, the ‘reference’ AE substrate is selected from the group consisting of ubiquitin and ubiquitin like proteins. In an embodiment of the invention, the ‘reference’ AE substrate is selected from the group consisting of Ub, NEDD8, ISG15, SUMO1, SUMO2, SUMO3, UFM1, FUBI, FAT10, Urm1, FAU, ATG8, ATG12, GABARAP, GABARAPL1, GABARAPL2, MAP1LC3A, MAP1LC3B, MAP1LC3B2 and MAP1LC3C. In an embodiment of the invention, the ‘reference’ AE substrate is selected from the group consisting of Ub, NEDD8, ISG15, SUMO1, SUMO2, SUMO3, UFM1, FUBI, FAT10, ATG8, ATG12 and URM1.

In a particularly preferred embodiment of the invention, AE substrate analogues are provided according to formula (I) as defined herein before wherein R′ represents hydrogen; X represents —NH— or —O—, preferably —NH—; and R represents a peptide selected from the group consisting of Ub(1-75), NEDD8(1-75), ISG15(1-156), SUMO1(1-96), SUMO2(1-92), SUMO3(1-91), UFM1(1-82), FUBI(1-73), FAT10(1-164); Urm1(1-100); FAU(1-73); ATG12(1-139); ATG8(1-115) GABARAP(1-115); GABARAPL1(1-115); GABARAPL2(1-115); MAP1LC3A(1-119); MAP1LC3B(1-119); MAP1LC3B2(1-119); and MAP1LC3C(1-125) or an N-terminally truncated variant thereof. In an embodiment R represents a peptide selected from the group consisting of Ub(1-75), NEDD8(1-75), ISG15(1-156), SUMO1(1-96), SUMO2(1-92), SUMO3(1-91), UFM1(1-82), FUBI(1-73) and FAT10(1-164) or an N-terminally truncated variant thereof.

Hence, in a preferred embodiment of the invention, the AE substrate analogue is selected from the group consisting of Ub(1-75)-ΔAla, NEDD8(1-75)-ΔAla, ISG15(1-156)-ΔAla, SUMO1(1-96)-ΔAla, SUMO2(1-92)-ΔAla, SUMO3(1-91)-ΔAla, Ufm1(1-82)-ΔAla, FUBI(1-73)-ΔAla, Fat10(1-164)-ΔAla, Urm1(1-100)-ΔAla; FAU(1-73)-ΔAla; ATG12(1-139)-ΔAla; GABARAP(1-115)-ΔAla; GABARAPL1(1-115)-ΔAla; GABARAPL2(1-115)-ΔAla; MAP1LC3A(1-119)-ΔAla; MAP1LC3B(1-119)-ΔAla; MAP1LC3B2(1-119)-ΔAla; and MAP1LC3C(1-125)-ΔAla; N-terminally truncated variants of said peptides comprising a number of amino acid residues of equal to or higher than 2, preferably equal to or higher than 3, more preferably equal to or higher than 5, and homologues of said peptides or N-terminally truncated variants thereof. In an embodiment of the invention, the AE substrate analogue is selected from the group consisting of Ub(1-75)-ΔAla, NEDD8(1-75)-ΔAla, ISG15(1-156)-ΔAla, SUMO1(1-96)-ΔAla, SUMO2(1-92)-ΔAla, SUMO3(1-91)-ΔAla, Ufm1(1-82)-ΔAla, FUBI(1-73)-ΔAla and Fat10(1-164)-ΔAla; N-terminally truncated variants of said peptides and homologues of said peptides or N-terminally truncated variants thereof.

Specific embodiments of the invention concern truncated versions of the AE substrate analogues of the invention. It will be understood by those skilled in the art that, for maintaining the capability of the (modified) AE substrate to be recognized by the AE active site, truncations of the N-terminal part of the (modified) substrate protein or peptide, may not affect recognition by and interaction with the AE. The length of any truncation is not particularly limited provided that the remaining peptide is still capable of being recognized by and interacting with the adenylating enzyme, and thereby act as an ABP and/or inhibitor. Suitable methods for establishing these characteristics are described herein elsewhere. As will be illustrated in the examples, the strategy of the present invention was successfully used to produce the peptide VYRFYG-ΔAla which is the N-terminally truncated ΔAla ubiquitin analogue.

In a preferred embodiment of the invention N-terminally truncated variants of the AE substrate analogues are provided having a length of at least 2 amino acid residues, preferably at least 3, more preferably at least 4, more preferably at least 5, more preferably at least 6, more preferably at least 7, more preferably at least 8, more preferably at least 9, more preferably at least 10, more preferably at least 12, more preferably at least 15, more preferably at least 20. In a preferred embodiment —R represents truncated versions of the corresponding portion of the ‘wild-type’ AE substrate, with the proviso that the resulting agent is still capable of being recognized by and interacting with the active site of the adenylating enzyme. Preferably, in the above formula —R represents an amino acid sequence having a length of at least 1, at least 2, at least 3, at least 4, at least 5, at least 6, at least 7, at least 9, at least 10, or at least 11, or at least 14, or at least 19 amino acid residues. In another embodiment, —R represents an amino acid sequence having a length of less than 25 amino acid residues, preferably less than 20, less than 15, less than 12, less than 10, less than 9, less than 8 or less than 7 amino acid residues.

In another preferred embodiment of the invention the AE substrate analogue is the full length AE substrate analogue. In another embodiment, the AE substrate analogue is an N-terminally truncated version containing at least 50%, at least 60%, at least 70%, at least 80%, at least 85%, at least 90%, at least 92.5%, at least 95%, at least 97%, at least 98% or at least 99% of the amino acid sequence of the full length (modified) AE substrate.

Specific embodiments of the invention concern homologues of an AE substrate that has been modified in accordance with the present invention. Hence, in an embodiment —R represents a homologue of the corresponding portion of the ‘wild-type’ AE substrate, with the proviso that the resulting agent is still capable of being recognized by and interacting with the active site of the adenylating enzyme.

The term ‘homologue’ is used herein in its common meaning, as referring to polypeptides which differ from a reference polypeptide, by minor modifications, but which maintain the basic polypeptide and side chain structure of the reference peptide. Such changes include, but are not limited to: changes in one or a few amino acid side chains; changes in one or a few amino acids, including deletions, insertions and/or substitutions; changes in stereochemistry of one or a few atoms; additional N- or C-terminal amino acids; and/or minor derivatizations, including but not limited to: methylation, glycosylation, phosphorylation, acetylation, myristoylation, prenylation, palmitation, amidation and/or addition of glycosylphosphatidyl inositol. Non-naturally occurring mutants of particular interest furthermore include mutants comprising certain unnatural amino acids, by insertions and/or substitutions.

As used herein, a homologue or analogue has either enhanced or substantially similar functionality as the naturally occurring polypeptide. A homologue herein is typically understood to refer to a polypeptide having at least 50%, more preferably at least 70%, preferably at least 80%, more preferably at least 90%, still more preferably at least 95%, still more preferably at least 98% and most preferably at least 99% amino acid sequence identity with the reference polypeptide, when optimally aligned.

Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. For instance, “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

In an embodiment of the invention, AE substrate analogues are provided as defined herein before, based on non-natural, short-peptide mimics, such as those described in the prior art referred to elsewhere herein. Examples thereof include ubiquitin-mimicking peptides, such as VWRFHGG; VQRYWGG and VYRFYGG, and Nedd8-mimicking peptides, such as VLQWFGG, VRLWFGG, VILTFGG and VRLMFGG. Hence, in a preferred embodiment of the invention, the AE substrate analogue is selected from the group consisting of VWRFHGΔAla; VQRYWGΔAla, VYRFYGΔAla, VLQWFGΔAla, VRLWFGΔAla, VILTFGΔAla and VRLMFGΔAla.

In an embodiment [PEPTIDE] represents a conjugate of the corresponding portion of the wild-type AE substrate with another peptide or protein, which may be conjugated in a linear or non-linear fashion, with the proviso that the capability of the resulting agent to be recognized by and interacting with the active site of the adenylating enzyme is retained. Such conjugates may be used to introduce or affect chemical or biological functionality, e.g. cell permeability enhancement, proteasome targeting, introduction of sites for directed chemical modifications (introduction of a so-called ‘ligation handle’), affinity tagging, etc. Preferred examples include addition of cell penetration enhancing peptide sequences such as (D-Arg)8, Tat and penetratin; addition of affinity tag peptide sequences, such as HA and His6; addition of a proteasome targeting handle such as L4.

In addition to conjugations with other peptides, the here described invention also entails AE substrate analogues comprising a derivative of the above defined structures, with ligands coupled to an amino acid side chain thereof and/or the N-terminus thereof. Such ligands may, in principle, be of any nature, including peptides or proteins, lipids, carbohydrates, polymers and organic or inorganic agents. The introduction of the ligand typically introduces or affects a particular biological or chemical function. Particularly interesting examples include the introduction of detectable labels and tags, introduction of electrophilic traps, introduction of chemical ligation moieties, etc. Hence, in a preferred embodiment, said derivative comprises a ligand selected from the group of fluorophores, affinity labels, biophysical labels, chelating agents, complexing agents and epitope tags, such as fluorescein, TAMRA or DOTA. Those skilled in the art will be familiar with these types of ligands and their introduction at a desired site can be accomplished using processe, reagents and conditions that are generally known.

The present invention also entails AE substrate analogues in the form of peptide mimetics comprising a spatial arrangement of (re)active chemical moieties and/or functional groups that resembles the three-dimensional arrangement of active and/or functional groups of any one of the AE substrate analogues defined herein before, wherein the peptide mimetic in any case comprises a C-terminal dehydroalanine moiety, or a comparable acrylic acid moiety, and wherein said peptide mimetic is capable of being recognized by and interacting with the active site of the adenylating enzymes, which capability can be established using the methods and assays described herein elsewhere. A peptide mimetic (peptidomimetic) is a molecule that mimics the biological activity of a peptide, yet is no longer peptidic in chemical nature. By strict definition, a peptidomimetic is a molecule that no longer contains any peptide bonds, i.e., amide bonds between amino acids; however, in the context of the present invention, the term peptide mimetic and also the term peptidomimetic are intended to include molecules that are no longer completely peptidic in nature, such as pseudo-peptides, semi-peptides and peptoids. Whether completely or partially nonpeptide, peptidomimetics according to the present invention provide a spatial arrangement of (re)active chemical moieties and/or functional groups that closely resembles the three-dimensional arrangement of active and/or functional groups in the peptide on which the peptidomimetic is based. The techniques of developing peptidomimetics are conventional. Thus, non-peptide bonds that allow the peptidomimetic to adopt a similar structure to the original peptide can replace peptide bonds. Replacing chemical groups of the amino acids with other chemical groups of similar structure and/or function (sometimes referred to as bio-iosteres) can also be used to develop peptidomimetics. Conventional approaches allow for the development of peptidomimetics in accordance with this invention.

The structures defined above, as will be evident from the teachings herein, are derived from AE substrates, modified by the introduction of a methylene moiety at the α carbon atom. It will be understood by those skilled in the art, that in a similar manner ABP's and/or inhibitors can be produced on the basis of other adenylating enzyme substrates. In certain embodiments of the invention, such AE substrate analogues are provided derived from O-succinylbenzoate (OSB), which is the natural substrate for the acyl-CoA synthetase MenE, an adenylate producing enzyme. These structures therefore function as ABP and/or inhibitors of MenE and similar adenylating enzymes.

Hence, in an embodiment of the invention, an AE substrate analogue is provided having the structure according to formula (I):

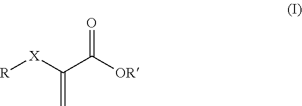

wherein R′ represents hydrogen or a moiety represented by the formula

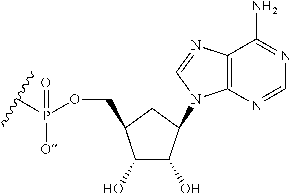

preferably R′ represent hydrogen; and wherein X represents —NH— or —O—, preferably —NH—; and wherein R represents —C(═O)—R^{x}, wherein R^{x} represent a ring structure selected from phenyl, pyridyl, pyrrolyl, cyclohexyl, cyclopentyl, pyranyl, furanyl, 1,2-dihydro-2-oxo-1H-pyrid-3-yl, and 2-oxopyranyl; which ring structure is optionally substituted with one or more substituents independently selected from hydroxy, amino, halo, cyano, nitro, —CF₃, —CHF₂, —CH₂F, trifluoromethoxy, azido, (C₁-C₆)alkoxy, (C₁-C₆)alkanoyloxy, (C₁-C₆)alkyl, (C₂-C₆)alkenyl and (C₂-C₆)alkynyl, (C₁-C₆)carboxyl, (C₁-C₆)carboxy-(C₁-C₆)alkyl, thiazolidine dione, hydroxamic acid, acyl-cyanamide, tetrazole, isoxazole, hydroxylisoxazole, oxadiazolone, phosphonate, sulphonate, and sulfonamide.

In a particularly preferred embodiment of the invention, R represents —C(═O)—Ar, wherein Ar represents aryl, preferably a phenyl ring, which is optionally substituted with one or more substituents independently selected from hydroxy, amino, halo, cyano, nitro, —CF₃, —CHF₂, —CH₂F, trifluoromethoxy, azido, (C₁-C₆)alkoxy, (C₁-C₆)alkanoyloxy, (C₁-C₆)alkyl, (C₂-C₆)alkenyl and (C₂-C₆)alkynyl, (C₁-C₆)carboxyl, (C₁-C₆)carboxy-(C₁-C₆)alkyl, thiazolidine dione, hydroxamic acid, acyl-cyanamide, tetrazole, isoxazole, hydroxylisoxazole, oxadiazolone, phosphonate, sulphonate, and sulfonamide. The term “aryl”, as used herein, means a 5-10 membered carbocyclic aromatic system containing one, two or three rings wherein such rings may be attached together in a pendant manner or may be fused. The term “aryl” embraces aromatic radicals such as phenyl, naphthyl, biphenyl, etc.

In a particularly preferred embodiment R represents —C(═O)—Ar, wherein Ar represents phenyl, which is optionally substituted with one or more substitutents independently selected from hydroxy, (C₁-C₆)alkoxy, (C₁-C₆)alkyl, (C₁-C₆)carboxyl and (C₁-C₆)carboxy-(C₁-C₆)alkyl, preferably (C₁-C₃)alkoxy, (C₁-C₃)alkyl, (C₁-C₃)carboxyl and (C₁-C₃)carboxy-(C₁-C₃)alkyl, most preferably hydroxy, methoxy, methyl, carboxy and —C(═O)O—CH₃.

In a particularly preferred embodiment of the invention R represents a structure according to formula (III)

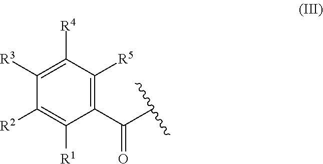

wherein
R⁵ is an optionally substituted carboxylic acid, carboxylate ester or a carboxylic acid isostere; R¹-R⁴ are independently selected from hydrogen, hydroxy, amino, halo, cyano, nitro, —CF₃, —CHF₂, —CH₂F, trifluoromethoxy, azido, (C₁-C₆)alkoxy, (C₁-C₆)alkanoyloxy, (C₁-C₆)alkyl, (C₂-C₆)alkenyl and (C₂-C₆)alkynyl, with the proviso that at least 2, preferably at least 3 of R¹-R⁴ represent hydrogen.

In the above formula, carboxylic acid denotes the group —R′—C(O)OH and carboxylate ester denotes the group —R′—C(O)O—R″, where R′ represents (C₁-C₆)alkyl or a covalent bond, preferably a covalent bond, and R″ preferably represents an optionally substituted (C₁-C₆)alkyl or (C₁-C₆)alkenyl, preferably a (C₁-C₃)alkyl, especially methyl or ethyl.

In functional terms, a “Carboxylic acid isostere” means a moiety that mimics carboxylic acids by virtue of similar physical properties, including but not limited to molecular size or molecular shape, thereby producing similar biological effects as those produced by a carboxylic acid group. “Carboxylic acid isostere” typically refers to a group selected from thiazolidine dione, hydroxamic acid, acyl-cyanamide, tetrazole, isoxazole, hydroxylisoxazole, oxadiazolone, phosphonate, sulphonate, and sulfonamide (The Practice of Medicinal Chemistry, Edited by Camille G. Wermuth, Second Edition, 2003, 189-214). Isoxazole may be optionally substituted with (C₁-C₃)alkyl, (C₁-C₃)alkyl substituted with 1-3 fluoro, aryl or heteroaryl, wherein aryl or heteroaryl may be optionally substituted with 1-3 groups or substituents selected from halo, (C₁-C₃)alkyl, fluoro substituted (C₁-C₃)alkyl, (C₁-C₃) alkoxy, fluoro substituted (C₁-C₃)alkoxy, (C₁-C₃)alkylthio, and fluoro substituted (C₁-C₃)alkylthio. Sulfonamide may be optionally substituted with (C₁-C₃)alkyl, fluoro substituted (C₁-C₃)alkyl, (C₁-C₃)acyl, aryl and heteroaryl, wherein aryl or heteroaryl may be optionally substituted with 1-3 groups or substituents selected from halo, (C₁-C₃)alkyl, fluoro substituted lower (C₁-C₃)alkyl, (C₁-C₃)alkoxy, fluoro substituted (C₁-C₃)alkoxy, lower (C₁-C₃)alkylthio, and fluoro substituted (C₁-C₃)alkylthio.

In a particularly preferred embodiment of the invention, R⁵ represents a moiety selected from the group consisting of —C(═O)—OH and —C(═O)—O—CH₃.

In a particularly preferred embodiment of the invention, R¹-R⁴ are independently selected from hydrogen, hydroxy, halo and (C₁-C₃)alkyl, with the proviso that at least 2, preferably at least 3 of R¹-R⁴ represent hydrogen. More preferably R¹-R³ represent hydrogen and R⁴ represents hydrogen or hydroxyl. Most preferably R¹-R⁴ represent hydrogen.

In a particularly preferred embodiment of the invention R represents a structure according to formula (IV)

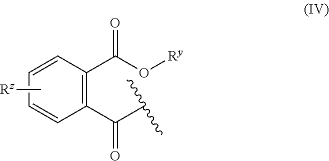

wherein
R^{Y} is selected from hydrogen and C₁-C₆ alkyl;
R^{z} is an optional substituent selected from hydroxy, amino, halo, cyano, nitro, —CF₃, —CHF₂, —CH₂F, trifluoromethoxy, azido, (C₁-C₆)carboxy, (C₁-C₆)alkoxy, (C₁-C₆)alkanoyloxy, (C₁-C₆)alkyl, (C₂-C₆)alkenyl and (C₂-C₆)alkynyl.

R^{Y} preferably represents hydrogen, methyl, ethyl or propyl, more preferably hydrogen or methyl, most preferably hydrogen.

R^{z} may be in the ortho, meta or para position, relative to the —C(═O)—O—R^{y} moiety. Preferably it is in the meta position. In a preferred embodiment R^{z} is selected from the group consisting of hydrogen, hydroxyl, (C₁-C₃)alkyl and halo. Preferably R^{z} is hydrogen or hydroxyl, most preferably it is hydrogen. In a particularly preferred embodiment of the invention a compound is defined above is provided wherein the optional substituent R^{z} is absent.

The structures defined above, as will be evident from the teachings herein, are derived from natural AE substrates, modified by the introduction of a methylene moiety at the α carbon atom. It will be understood by those skilled in the art, that in a similar manner ABP's and/or inhibitors can be produced on the basis of other adenylating enzyme substrates. In certain embodiments of the invention, such AE substrate analogues are based on the modification, according to the teachings of this invention, of natural substrates of adenylating enzymes selected from the group consisting of fatty acyl-CoA synthetases (FACS) (Exp Biol Med, 2008 vol. 233, 507). These ligases form thioester linked fatty acid-coenzyme A conjugates by first forming a fatty acid-AMP intermediate (ATP dependant step) and subsequently transferring the fatty acid to the thiol of coenzyme A. By elongation, degradation, or incorporation of fatty acids into complex lipids, FACS are believed to regulate various biological processes (Ellis et al. Curr Opin Lipidol 2010, Vol 21, 212). Examples of FACS substrates are caprylic acid (C8), capric acid (C10), lauric acid (C12), myristic acid (C14), palmitic acid (C16), stearic acid (C18).

Hence, in an embodiment of the invention, an AE substrate analogue is provided having the structure according to formula (I):

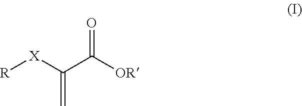

wherein R′ represents hydrogen or a moiety represented by the formula

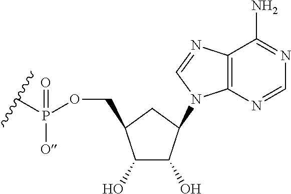

preferably R′ represent hydrogen; and wherein X represent —CH₂—; and wherein R represents a fatty acid tail remnant, preferably R represents a C_{ω}-C_{γ} aliphatic saturated or mono- or polyunsaturated carbon atom chain, identical to the C_{ω}-C_{γ} part of an AE substrate fatty acid, wherein the carbon atoms are designated relative to the fatty acid carboxylate group, C_{α} representing the carbon atom adjacent to the carboxylate carbon atom of the fatty acid and C_{ω} represents the terminal carbon atom of the fatty acid tail.

In an embodiment of the invention, said AE substrate analogues are based on natural substrates of fatty acyl-CoA synthetases as already mentioned here above. Suitable examples thereof include myristoleic acid; palmitoleic acid; sapienic acid; oleic acid; elaidic acid; vaccenic acid; linoleic acid; linoelaidic acid; α-linolenic acid; arachidonic acid; eicosapentaenoic acid; erucic acid; docosahexaenoic acid; caprylic acid; capric acid; lauric acid; myristic acid; palmitic acid; stearic acid; arachidic acid; behenic acid; lignoceric acid; and cerotic acid.

Hence, in an embodiment of the invention, AE substrate analogues are provided according to formula (I) as defined herein before wherein R′ represents hydrogen; X represents —CH₂—; and R represents a moiety selected from the group consisting of
CH₃(CH₂)₃CH═CH(CH₂)₅—;
CH₃(CH₂)₅CH═CH(CH₂)₅—;
CH₃(CH₂)₈CH═CH(CH₂)₂—;
CH₃(CH₂)₇CH═CH(CH₂)₅—;
CH₃(CH₂)₇CH═CH(CH₂)₅—;
CH₃(CH₂)₅CH═CH(CH₂)₇—;
CH₃(CH₂)₄CH═CHCH₂CH═CH(CH₂)₅—;
CH₃(CH₂)₄CH═CHCH₂CH═CH(CH₂)₅—;
CH₃CH₂CH═CHCH₂CH═CHCH₂CH═CH(CH₂)₅—;
CH₃(CH₂)₄CH═CHCH₂CH═CHCH₂CH═CHCH₂CH═CHCH₂—;
CH₃CH₂CH═CHCH₂CH═CHCH₂CH═CHCH₂CH═CHCH₂CH═CHCH₂—;
CH₃(CH₂)₇CH═CH(CH₂)₉—;
CH₃CH₂CH═CHCH₂CH═CHCH₂CH═CHCH₂CH═CHCH₂CH═CHCH₂CH═CH—;
CH₃(CH₂)₄—
CH₃(CH₂)₆—;
CH₃(CH₂)₈—;
CH₃(CH₂)₁₀—;
CH₃(CH₂)₁₂—;
CH₃(CH₂)₁₄—;
CH₃(CH₂)₁₆—;
CH₃(CH₂)₁₈—;
CH₃(CH₂)₂₀—; and
CH₃(CH₂)₂₂—.

Hence, in a preferred embodiment of the invention, the AE substrate analogue according to formula (I) is selected from the group consisting of:
CH₃(CH₂)₃CH═CH(CH₂)₆—C(═C)—COOH;
CH₃(CH₂)₅CH═CH(CH₂)₆—C(═C)—COOH;
CH₃(CH₂)₈CH═CH(CH₂)₃—C(═C)—COOH;
CH₃(CH₂)₇CH═CH(CH₂)₆—C(═C)—COOH;
CH₃(CH₂)₇CH═CH(CH₂)₆—C(═C)—COOH;
CH₃(CH₂)₅CH═CH(CH₂)₈—C(═C)—COOH;
CH₃(CH₂)₄CH═CHCH₂CH═CH(CH₂)₆—C(═C)—COOH;
CH₃(CH₂)₄CH═CHCH₂CH═CH(CH₂)₆—C(═C)—COOH;
CH₃CH₂CH═CHCH₂CH═CHCH₂CH═CH(CH₂)₆—C(═C)—COOH;
CH₃(CH₂)₄CH═CHCH₂CH═CHCH₂CH═CHCH₂CH═CH(CH₂)₂—C(═C)—COOH;
CH₃CH₂CH═CHCH₂CH═CHCH₂CH═CHCH₂CH═CHCH₂CH═CH(CH₂)₂—C(═C)—COOH;
CH₃(CH₂)₇CH═CH(CH₂)₁₉—C(═C)—COOH;
CH₃CH₂CH═CHCH₂CH═CHCH₂CH═CHCH₂CH═CHCH₂CH═CHCH₂CH═CHCH₂—C(═C)—COOH;
CH₃(CH₂)₅—C(═C)—COOH;
CH₃(CH₂)₇—C(═C)—COOH;
CH₃(CH₂)₉—C(═C)—COOH;
CH₃(CH₂)₁₁—C(═C)—COOH;
CH₃(CH₂)₁₃—C(═C)—COOH;
CH₃(CH₂)₁₅—C(═C)—COOH;
CH₃(CH₂)₁₇—C(═C)—COOH;
CH₃(CH₂)₁₉—C(═C)—COOH;
CH₃(CH₂)₂₁—C(═C)—COOH; and
CH₃(CH₂)₂₃—C(═C)—COOH.

In a preferred embodiment of the invention, a modified adenylating enzyme substrate as defined here above is provided, wherein X represents —CH₂—; and —R represents a saturated, aliphatic CH₃—(CH₂)ₙ— chain, wherein n represents 4, 6, 8, 10, 12, 14, 16, 18, 20, 22 or 24.

In a preferred embodiment, the invention in particular does not encompass any one of the following compounds:
(RS)-2-[2-(3-carboxy-1-(2-methylphenyl)propoxy)-4-(3-thienylmethoxy)benzoylamino]-acrylic acid;
COOH—C(═C)—(CH₂)₂—C(═C)—COOH;
COOH—CH₂—C(═C)—COOH;
COOH—(CH₂)₂—C(═C)—COOH;
COOH—(CH₂)₃—C(═C)—COOH; and
COOH—(CH₂)₄—C(═C)—COOH.

The compounds defined here above may contain one or more chiral centers and/or double bonds and, therefore, exist as stereoisomers, such as double-bond isomers (i.e., geometric isomers), enantiomers, or diastereomers. According to the invention, the chemical structures as depicted herein, and therefore the compounds of the invention, encompass all of the corresponding compound's enantiomers and stereoisomers, that is, both the stereomerically pure form (e.g., geometrically pure, enantiomerically pure, or diastereomerically pure) and enantiomeric and stereoisomeric mixtures.

The invention also concerns pharmaceutically acceptable salts, esters, solvates and hydrates of the compounds of the present invention. Such salts include inorganic and organic acid or base addition salts of compounds of the present invention. These salts can be prepared in situ during the final isolation and purification of the compounds or by separately reacting the purified compound in its free base form with a suitable organic or inorganic acid and isolating the salt thus formed. Examples of pharmaceutically acceptable base addition salts include sodium, potassium, calcium, ammonium, organic amino, or magnesium salt, or a similar salt. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Examples of suitable esters include compounds of the invention which hydrolyze in vivo and include those that break down readily in the human body to leave the parent compound or a salt thereof. Examples of pharmaceutically acceptable, relatively nontoxic esters of the invention include (C₁-C₆)alkyl esters and (C₅-C₇)cycloalkyl esters. Esters of the compounds of the invention can be prepared according to conventional methods. Pharmaceutically acceptable esters can be obtained through reaction of hydroxy groups of the compound with an organic acid, such as acetic acid or benzoic acid. In the case of compounds containing carboxylic acid groups, the pharmaceutically acceptable esters are prepared by reaction of said carboxylic acid group, as will be understood by those skilled in the art.

Another aspect of the present invention concerns a method of producing a AE substrate analogue comprising the steps of:
selecting an adenylating enzyme of interest;
selecting a natural or non-natural substrate or ligand for the adenylating enzyme comprising a C-terminal carboxylate in the moiety capable of interacting with the adenylating enzyme;
modifying the C-terminal carboxylate moiety of said adenylating enzyme substrate by introducing a methylene group at the carbon atom in the α-position relative to the C-terminal carboxylate group.

As will be understood by those skilled in the art, the substrate for the AE typically will be a or the natural substrate for the adenylating enzyme, examples of which have been described herein elsewhere.

In a particularly preferred embodiment of the present invention, a method as defined herein before is provided, wherein the AE substrate is a peptide or protein, said method comprising the steps:
selecting an adenylating enzyme of interest;
selecting a natural substrate peptide or protein for the adenylating enzyme;
chemical and/or biological synthesis of the selected peptide substrate, wherein said synthesis comprises substituting the C-terminal amino acid residue of the selected natural substrate peptide or protein by a dehydroalanine residue.

Chemical peptide synthesis methods are well known to the person skilled in the art. In accordance with the present invention the peptides are typically chemically synthesized, preferably using solid phase synthesis. In accordance with the present invention, it is not critical whether the entire sequence is synthesised through stepwise elongation only or whether the process involves ligation of two or more separately obtained fragments. The synthesis of the peptide may be performed on a solid phase substrate, yielding a peptide that is covalently attached to said substrate.

The incorporation of the dehydroalanine residue may be accomplished in various ways, which as such, are common general knowledge for those of average skill in the art. In a preferred embodiment of the invention an (alkylated) cysteine residue is incorporated, substituting the C-terminal residue of the ‘wild-type’ peptide sequence, which (alkylated) cystein residue is subsequently converted into the dehydroalanine moiety by oxidative elimination, e.g. with O-mesitylenesulfonylhydroxylamine (MSH).

One or more of the subsequent steps of the present method may be performed before or after release of the peptide from said solid phase substrate. The method of the invention may concern solid phase synthesis without release from the solid phase substrate prior to or after the subsequent steps of the invention, e.g. in the case of protein (micro)arrays, where the protein can be synthesized directly on the microarray surface.

Furthermore, as will be clear from the foregoing, the method may comprise additional modifications of the AE substrate, e.g. by truncations, derivatizations, conjugations, amino acid deletions, insertions or substitutions, etc., with the proviso that the capability of the resulting structure to be recognized by and interact with the active site of the AE is retained.

The present invention also entails the production of AE substrate analogue peptides and proteins using an expression system, a method which is generally known and understood by those skilled in the art. When using an expression system, a mutant of the wild-type AE substrate is produced, wherein the C-terminal amino acid residue is substituted with a cysteine residue. As explained here above, the cysteine residue can subsequently be (chemically) converted into the dehyrdoalanine moiety to provide the analogue according to the invention.

As will be understood, particularly preferred features described here above in relation to the AE substrate analogues, apply mutatis mutandis to the method of producing them.

Another aspect of the invention concerns AE substrate analogues obtainable by the afore-defined methods.

Another aspect of the present invention concerns the use of the AE substrate analogues as defined in any of the foregoing as a medicament, a diagnostic agent and/or as biochemistry research tool.

As will be understood by one skilled in the art the AE substrate analogues of the present invention can be used to capture a corresponding adenylating enzyme, e.g. from a highly complex biological matrix, which can be of particular use in both diagnostics and fundamental research.

Hence, the invention, in one aspect, also provides a method of capturing an adenylating enzyme from a biological sample, said method comprising the steps of: a) providing said sample comprising an adenylating enzyme; b) combining the sample with a corresponding AE substrate analogue of this invention, wherein said AE substrate analogue is conjugated to a chelating agent, a complexing agent, an epitope tag or a solid phase, which allows for or results in immobilization of the AE substrate analogue agent; c) subjecting the sample to conditions that allow for selective binding of the adenylating enzyme to the AE substrate analogue; d) separating the sample from the immobilized AE substrate analogue. As will be understood by those skilled in the art, the irreversible binding of the AE substrate analogue to a corresponding adenylating enzyme requires the presence of ATP. Hence, in an embodiment step a), b) or c) comprises the addition of ATP to the sample. ATP added to the sample, in accordance with the invention may be isotopically labeled ATP, e.g. [³²P]ATP, to facilitate the detection of ‘trapped’ AE substrate.

Immobilization of AE substrate analogues, can be achieved using various techniques familiar to those skilled in the art. Depending on the choice of immobilization technique the above-described method may comprise the additional step of combining the sample comprising the AE substrate analogue with a solid phase capable of immobilizing the AE substrate analogue, prior to any one of steps a), b), c) or d). If the immobilization step is done after step b), as will be understood, a technique is to be selected involving selective trapping under condition which do not affect other components of the biological sample. Hence, it will be appreciated that a preferred embodiment of the method comprises immobilization of the AE substrate analogue prior to step b). As will be understood by those skilled in the art, immobilization of the AE substrate analogue can be accomplished in various ways. In one embodiment of the invention, the AE substrate analogue is immobilized using CNBr-activated sepharose.

In an embodiment of the invention, the above method involves the use of a AE substrate analogue that is conjugated/derivatized with a detection label as defined herein above, wherein the method comprises one or more additional steps of quantifying the binding of adenylating enzyme to the AE substrate analogue.

The present AE substrate analogues are capable of binding a corresponding adenylating enzyme in a selective and highly irreversible manner, allowing for stringent washing conditions, which makes the present method highly effective.

The above method may be used in research concerning any biological process involving the action of an adenylating enzyme and/or in diagnosing any condition or disease involving the action of an adenylating enzyme. Hence, an aspect of the invention concerns the use of a AE substrate analogue as defined herein, for capturing adenylating enzyme in a biological matrix in vitro or ex vivo. Another aspect of the invention concerns a AE substrate analogue as defined herein for use in a diagnostic method.

Since the present modified adenylating enzyme substrates are capable of selective and highly irreversible binding of their corresponding adenylating enzyme, it is also envisaged that they have utility as (competitive) AE inhibitors or as antagonistic agents in various therapeutic methods. Typically such therapeutic methods are aimed at the treatment or prevention of a condition or disease, involving the action of an adenylating enzyme. Hence, an aspect of the invention concerns the use of a AE substrate analogue as defined herein, for use in a method of therapeutic and/or prophylactic treatment in a subject in need thereof. Another aspect of the invention concerns a AE substrate analogue as defined herein for use in a method of treating and/or preventing a disease involving the action of an adenylating enzyme in a subject in need thereof and/or a disease involving a pathway involving an adenylating enzyme.

In an embodiment of the invention, said disease is a disease involving the ubiquitin or a ubiquitin-like conjugation pathway. As explained herein before, AE substrate analogues based on ubiquitin or ubiquitin-like proteins are actually capable of targeting the entire E1-E2-E3 machinery. Hence in an embodiment of the invention, the method comprises the administration of such an AE substrate analogue to inhibit the action of an E1 enzyme, an E2 enzyme or an E3 enzyme, in particular HECT type E3 enzyme or RBR type E3 enzyme.

The invention, in further aspects, provides the use of a modified adenylating enzyme substrate as defined in the foregoing as an inhibitor or antagonist of a corresponding adenylating enzyme; a method of inhibiting adenylating enzyme activity by exposing the adenylating enzyme to a corresponding AE substrate analogue as defined herein before; and the AE substrate analogue for use in any such method.

Thus, the invention has been described by reference to certain embodiments discussed above. It will be recognized that these embodiments are susceptible to various modifications and alternative forms well known to those of skill in the art.

Many modifications in addition to those described above may be made to the structures and techniques described herein without departing from the spirit and scope of the invention. Accordingly, although specific embodiments have been described, these are examples only and are not limiting upon the scope of the invention.

Furthermore, for a proper understanding of this document and in its claims, it is to be understood that the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### EXPERIMENTAL

### Introduction

Ubiquitin (Ub) and ubiquitin like (Ub1) proteins regulate a wide variety of key biological processes by covalently modifying protein substrates. The covalent conjugation of Ub/Ub1 proteins is controlled by a tandem action of E1, E2 and E3 enzymes. Initial steps are catalyzed by an E1 (activating) enzyme in two steps. First, the E1 uses ATP and Mg²⁺ to adenylate the C-terminus of Ub/Ub1 to form a Ub/Ub1-AMP intermediate, releasing pyrophosphate. Secondly, the C-terminal Ub/Ub1 adenylate is attacked by the catalytic Cys in the E1 to form the activated E1˜Ub/Ub1 thioester, resulting in release of AMP. Subsequently an E2 binds the E1˜Ub/Ub1 thioester triggering Ub transfer from the E1 to a conserved cysteine residue of the E2, forming an E2˜Ub/Ub1 thioester intermediate. Finally, the Ub/Ub1 is transferred to the target protein by the action of an E3 ligase.

Ub/Ub1 modification can be reversed by the action of deubiquinating enzymes (DUBs), a class of proteases that cleave Ub/Ub1 proteins from substrates. DUBs are studied extensively because of their role in regulating many biological processes. An important class of reagents used to study DUB activity and substrate specificity are ubiquitin based activity-based probes (ABPs). For the full Ub/Ub1 conjugation machinery (E1, E2, E3 enzymes) these tools are not available. Only recently SUMO and Ub E1 probes have been reported that contain a sulfamide as a nonhydrolyzable analogue of the phosphate in the Ub/Ub1˜AMP intermediate and a vinyl sulphonamide electrophile to trap the E1. However, after entrapment of the E1, the Ub/Ub1 cannot be processed further by the E2 enzyme, prohibiting the labeling of E2 and E3 binding partners.

Although the structures of several E1, E2 and E3 enzymes have been reported, outstanding questions remain about the mechanism of these conjugation reactions. To investigate these questions, a probe for the full conjugation machinery was to be developed. The design of a Ub/Ub1 protein that contains the native Ub structure required for processing by the E1-E2-E3 machinery was envisioned but at the same time also bears an electrophilic trap that does not hamper transfer to an E2 and E3. As such, the development of the Ub(1-75)-ΔAla structure (ΔAla=dehydroalanine) is presented here, which allows covalent labeling of the E1-E2-E3 enzyme classes of the Ub/Ub1 conjugation machinery (i.e. FIG. 1b). This downstream labeling is possible by the dual role of the ΔAla moiety as a Gly76 mimic and an in-situ generated electrophile. First, Ub(1-75)-ΔAla is converted with ATP by E1 into an adenylate. Next, there are two possible outcomes: 1. the E1 active site cysteine residue reacts with the activated methylene group of the ΔAla moiety, inactivating the E1 (pathway a, FIG. 1b); 2. the E1 active site cysteine residue follows the native route and attacks the carbonyl of the AMP ester. The probe is now charged onto the E1 as an active-site thioester and because of its native character this can be transferred to an E2. During this transfer the same two earlier described outcomes are possible: the E2 active site cysteine forms a covalent adduct with the probe (pathway a, FIG. 1b) or forms a native Ub-E2 thioester intermediate. Finally, the same is true for the E2 to E3 transfer, explaining how the probe design allows it to “travel” through the E1-E2-E3 machinery.

### Results & Discussion

There are various methods for the synthesis of the dehydroalanine group. For example, (alkylated) cysteine can be transformed into ΔAla by oxidative elimination with O-mesitylenesulfonylhydroxylamine (MSH). Thus, treatment of a solution of Ub(1-75)-Cys(Bn)-OMe (synthesised chemically) in sodium phosphate buffer pH 8 with 10 eq. MSH, gave according to LC-MS full conversion into Ub(1-75)-ΔAla-OMe within 2 hrs. Finally, the methyl ester was hydrolyzed at pH 10 within 90 minutes (FIG. 2) to afford the ΔAla group. Another method employs 2,5-dibromohexandiamide as reagent for ΔAla formation from cysteine: here a 0.17 mM solution of Ub(1-75)-Cys-OH in 100 mM sodium phosphate pH 8 is incubated overnight at 37° C. with 10 eq of 2,5-dibromohexandiamide (1.7 mM). In contrast to MSH, the 2,5-dibromohexandiamide tolerates a free carboxylic acid next to the thiol.

Next the reactivity of the E1 Uba1 with Ub(1-75)-ΔAla and formation of the covalent bond between the probe and active site cysteine of the E1 was investigated. After incubation of Uba1 with Ub(1-75)-ΔAla under native conditions, SDS-PAGE analysis showed formation of a band above the Uba1 band, corresponding to an Uba1-Ub conjugate (FIG. 8a). Treatment of the reaction mixture with 2-mercaptoethanol under heating had no effect on the formed conjugate (FIG. 8), in line with the expected formation of the stable thioether linkage. When Uba1 was incubated with the probe in the absence of ATP, no labeling was observed, demonstrating that Ub(1-75)-ΔAla is processed and activated as in the native ATP dependent manner. The same observations were made in a labeling experiment with Uba6 (FIG. 8b), the second Ub E1 enzyme (which also activates FAT10).

Subsequently, the E2 class of enzymes was evaluated. Incubation of Ub(1-75)-ΔAla with E1, ATP and the E2 Ubc7 resulted in the formation of an Ub(1-75)Ala-E2 conjugate. The labelling was compared to a native situation with Ub (FIG. 9). Under non-reducing conditions both the Ub and Ub(1-75)-ΔAla probe showed labeling, whereas under reducing conditions only the probe could form a stable conjugate with the E2. Again, no labeling was observed in the absence of ATP. Specificity and potential utility of the probe was then tested with a panel of E2 enzymes (FIG. 10). Labeling reactions were performed with or without ATP for 2 h at 30° C. in the presence of UBE1, and terminated by the addition of reducing sample buffer and analyzed by SDS-PAGE. Even under these non-optimized conditions, labeling of 27 different E2s was observed (FIG. 10), which confirmed broad utility and selectivity of the probe. Importantly, the negative control reactions with E2s UBE2F, UBE2I, UBE2L6 and UBE2M showed no labeling because they recognize Ub1s other than Ub, and the Ub E2 UBE2Z was also inactive with the E1 UBA1 due to its selectivity for the alternative E1, UBA6. The controls UBE2V1 and UBE2V2 also showed no labeling since they belong to a distinct subfamily within the E2 protein family of noncanonical E2s, known as ubiquitin-conjugating enzyme variant (UEV) proteins, that perform a scaffolding role and are catalytically inactive (FIG. 10). Thus, this new ligase probe labels many E2s specific for Ub transfer but not E2s employing Ub1s, in line with their native mechanism based mode of action.

Having established the labeling conditions of the E1 and E2 enzymes, the E3 ligases were targeted. These enzymes are classified into three classes according to domain homology and mechanism of action. The HECT (homologous to E6-AP terminus) and RING-between-RING (RBR) classes have the same thioester loading mechanism of the E1 and E2 enzymes (i.e. a two-step reaction in which ubiquitin is transferred from the E2 to an active site cysteine in the E3 and then from the E3 to the substrate). This in contrast to the RING (Really Interesting New Gene) E3 ligases, which have large binding interfaces and act as scaffold proteins that bring together the Ub-E2 thioester and substrate protein.

To evaluate the labelling efficiency of probe in the case of the E3 ligases, first the well-characterized HECT E3 ligase Nedd4L was examined.

Incubation of Nedd4L with Cy5-Ub(1-75)-ΔAla, ATP, UBE1 and UBE2L3 resulted in formation of an Ub-E3 thioether adduct, as judged by in-gel fluorescence scanning (FIG. 11). To determine if the probe can generally be used to monitor HECT E3s, labeling against a panel of nine HECT E3 ligases was evaluated (FIG. 12). Incubation with Ub(1-75)-ΔAla, ATP, UBE1 and UBE2D2 for 1 h at 30° C. resulted in the formation of an Ub-E3 thioether adduct as analyzed by SDS-PAGE (FIG. 12). As anticipated, E3 enzymes harboring an active site Cys residue were reactive towards the probe. In line with the notion that the labeling of E3 enzymes needs an active E1-E2 cascade, ATP was required for labeling (FIG. 12).

Both solution-based and crystallographic studies were performed to verify the structural integrity of the thioether-linked adducts. First, a large-scale conjugation of Ub(1-75)-ΔAla to ¹⁵N-labeled UBE2N was performed. Solution properties of the oxyester-linked UBE2N—O˜Ub conjugate (in which the active site Cys has been mutated to Ser) have been thoroughly characterized by NMR spectroscopy and small angle X-ray scattering, allowing us to validate the thioether linkage as a suitable mimic. Following purification of the UBE2N-AlaUb thioether adduct by size exclusion chromatography, the ¹H, ¹⁵N HSQC spectrum was recorded for comparison to UBE2N alone (FIG. 13a). Plotting chemical shift perturbations arising from conjugation with Ub highlighted several regions affected in both the thioether- and oxyester-linked samples (FIG. 13b; compare gray to black). When mapped onto the UBE2N structure, these commonly affected resonances primarily localized to Loop 3, Helix 2, Loop 8, and the penultimate C-terminal helix (FIG. 13c). These perturbed regions can be attributed to the “closed” conformation of the UBE2N-AlaUb thioether adduct, which is predominant in solution and indicates the overall behavior of the thioether-linked adduct to be similar to the oxyester linkage. Based on the magnitude of the chemical shift perturbation and the behavior in the HSQC spectrum, certain resonances in the thioether-linked UBE2N-AlaUb spectrum displayed markedly different characteristics from the oxyester-linked sample (FIG. 13b). In the UBE2N structure, these uniquely behaving resonances map to the region directly surrounding the active site (FIG. 13c,). Given the exquisite sensitivity of amide resonances to their local chemical environment, such differences between the two linkage types are most likely due to their differing chemical properties, and not to a larger structural change.

While solution studies indicate normal inter-domain behavior within the thioether-linked adduct, they lack the atomic resolution of the linkage itself or the surrounding active site residues. To obtain a high-resolution crystal structure, milligram quantities of UBE2D3-AlaUb thioether adduct were prepared and purified to homogeneity by repeated cation exchange, followed by size exclusion chromatography. The thioether-linked adduct readily crystallized under conditions published for the oxyester-linked UBE2D3-O˜Ub conjugate, and its structure was determined to 2.2 Å resolution by molecular replacement (Table 1). The overall UBE2D3-AlaUb thioether structure was strikingly similar to the published oxyester structure (PDB 3UGB), with C α RMSD values of 0.23 and 0.35 Å for UBE2D3 and Ub, respectively (FIG. 14a). The only significant deviation between the two structures was found in the Ub C-terminus near the linkage itself, manifested in an RMSD of 1.13 Å for Ub residues 73-76. The thioether linkage was readily revealed in the corresponding electron density (FIG. 14b), although detailed features of the omit map did suffer from high B factors in the flexible Ub C-terminus (average B-factor of 105.7 for Ub residues 75-76 compared to 49.4 for all protein). Nearby residues within the UBE2D3 active site adopt nearly identical conformations, with the only exception being Arg90, which was missing from the electron density (FIG. 14c). An overlay of the oxyester and thioether structures suggests that the additional carboxylate group of the thioether linkage could displace the Arg side chain from the E2 active site cleft, although to the inventors' knowledge there is currently no known role for this residue in E2 catalysis.

Although they are a close mimic of the native thioester linkage, oxyester-linked E2˜O-Ub/E2˜O-Ub1 conjugates remain susceptible to hydrolysis, particularly in the presence of an active E3 ligase. In contrast, a time course experiment showed that the UBE2D3-AlaUb and UBE2N-AlaUb thioether-linked adducts remained inert in the presence of activating factors such as the E3 ligases TRAF6 (RING-type) or NEDD4L (HECT-type), or in the presence of the accessory E2-variant UBE2V2 with or without TRAF6, respectively (FIG. 15). Like other catalytically inert mimics of native thioester-linked conjugates, the thioether-linked adduct should act as competitive inhibitor of the ligation machinery. To test this, a single-turnover assay was employed in which a native thioester linked UBE2N˜Ub conjugate was generated by co-incubation of UBE1, UBE2N, Ub, and ATP/Mg²⁺. The activation reaction was then quenched by treatment with apyrase, and activating factors UBE2V2 and cIAP were added to stimulate the formation of diUb (FIG. 16, left). Due to the large excess of monoUb to serve as the acceptor, diUb chains were the primary product of the reaction and serve as a good indicator of normal UBE2N/UBE2V2/cIAP activity. As increasing amounts of the nonhydrolyzable thioether-linked UBE2N-AlaUb mimic were titrated into the reaction conditions, levels of diUb product formation diminished, indicating a competition for the accessory UBE2V2/cIAP enzymes (FIG. 16, right). Combined with solution and crystallographic data, these functional assays support the utility of thioether-linked E2-AlaUb adducts as stable mimics in both structural and functional studies.

A very important application of activity-based probes is protein activity profiling in complex biological samples such as cell lysates or live cells. After validation of both the activity and structural integrity of the Ub(1-75)-ΔAla probes activity in cells was profiled. Four different cell lysates were incubated with increasing amounts of Cy5-Ub(1-75)-ΔAla, and labeled enzymes were visualized by in-gel fluorescence scanning (FIG. 17) showing clear dose-dependent labeling patterns for all cell lines.

Next, the probe was evaluated in living cells. Electroporation of Cy5-Ub(1-75)-ΔAla into HeLa cells revealed both nuclear and cytoplasmic distribution of the probe, similar to that observed for fluorescently labeled wild type Ub (FIG. 18a). Some cells harboring the probe were at various mitotic phases, suggesting that the probe could be used to follow enzymatic activity during cell division (FIG. 18b). To evaluate whether a specific enzymatic activity can be visualized inside living cells, Cy5-Ub(1-75)-ΔAla was introduced into Hela cells exogenously expressing either free GFP or GFP-tagged USE1—an E2 enzyme associated to the membrane of the endoplasmic reticulum. In free GFP expressing cells, Cy5-Ub(1-75)-ΔAla was once again freely dispersed throughout the cytoplasm and nucleosol (FIG. 18c, left panels). By contrast, in cells overexpressing USE1-GFP, the probe accumulated predominantly at GFP-positive membrane compartments (FIG. 18c, middle panels). As evidenced by pixel analysis, the highest signal intensity quadrant contained the majority of dual positive signal in USE1 expressing cells. Similarly, total quantification of pixel overlap revealed striking colocalization of Cy5-Ub(1-75)-ΔAla with USE1-GFP but not with GFP alone. Collectively, these experiments demonstrate that Cy5-Ub(1-75)-ΔAla can be used to interrogate ubiquitin ligase activity in cells.

To show that the probe design is generally applicable to the Ub1 family Nedd8 and SUMO based probes were synthesized. For the Nedd8 probe, we treated Nedd8 G76C mutant with 2,5-dibromohexanediamide to afford Nedd8(1-75)-ΔAla. After incubation of Nedd8(1-75)-ΔAla with Uba3/NAE1 under standard conditions, SDS-PAGE analysis under reducing conditions revealed the formation of the Uba3-Nedd8 thioether adduct and a double Nedd8 loaded Uba3 adduct (FIG. 19A). Incubation of the E2 UBE2M with Nedd8(1-75)-ΔAla, Uba3/NAE1 and ATP resulted in the formation of a Nedd8-UBE2M thioether adduct, as evidenced by SDS-PAGE analysis (FIG. 19A). Interestingly, as with the Ub E1 UBA1, treatment with 2-mercaptoethanol had no effect on adduct formation, no labeling was observed in the absence of ATP, and yet an additional higher running band could be observed in the E1 labeling reaction, presumably corresponding to one Nedd8(1-75)-ΔAla marking the active site Cys and the other bound to the adenylation domain, mimicking an E1 double-loaded intermediate. The formation of double Nedd8 conjugated Uba3 was suppressed when UBE2M was present during the labeling experiment. This may indicate transfer of Nedd8 probe to a nearby Cys in the adenylation domain of Uba3, which simply does not occur when Nedd8ΔAla is quickly transferred to the next step in the cascade, here transfer to E2.

Following the same principle of oxidative Cys elimination, we synthesized the SUMO probes SUMO1(1-96)-ΔAla, SUMO2(1-92)-ΔAla and SUMO3(1-91)-ΔAla. Note that the native Cys residue in SUMO1 (i.e. Cys52), SUMO2 (i.e. Cys48) and SUMO3 (i.e. Cys47) is mutated to a Ser residue. After incubation of these probes with SUMO E1 (SAE1/SAE2), SDS-PAGE analysis under reducing conditions revealed the formation of the expected E1-SUMO thioether adduct (FIG. 20).

Recently short peptides have been identified that mimic the C-terminal tail of Ub and can be processed in a native manner by the E1, E2, and E3 enzymes. The peptides were found to be competitive inhibitors of Ub loading onto the E1 enzyme, although their affinity for the E1 active site is significantly lower than that of Ub, resulting in IC₅₀ values of more than 100 μM. It was hypothesized that replacement of the C-terminal Gly of the peptides with a ΔAla group would result in a more effective inhibitor, since the ΔAla group would allow for a covalently crosslink with the active site cysteine of the E1. The most potent E1 inhibitor VYRFYGG and the analogues VYRFYG-ΔAla were selected and prepared. The peptides were incubated in varying concentrations with Uba1 and TAMRA-Ub and the extent of (TAMRA-Ub)-E1 formation was followed by fluorescent in-gel scanning. In case of an inhibitory effect of these peptides the formation of Ub-E1 conjugates would decrease. Initial velocities of (TAMRA-Ub)-E1 formation were measured at 15 seconds. Here both peptides showed to have an inhibitory effect (FIG. 21 and FIG. 22). However, when the reactions were allowed to proceed for 15 min, both peptides showed a major difference in inhibition profile. Whereas the VYRFYGG peptide showed no detectable inhibition of E1 activity anymore, the VYRFYG-ΔAla peptide showed a prolonged inhibition of the E1. Ultimately these peptides will be used as a starting point for the solid-phase synthesis of a ΔAla peptide library for the development of potent and selective E1 inhibitors.

To date, disruption of the Ubiquitin Proteasome System (UPS) has proven to be successful in the clinic (leukemia, multiple myeloma, and some lymphomas) by using proteasome inhibitors (e.g. Velcade/bortezomib and the recently FDA approved Kyprolis/carfilzomib). Targeting the E1-E2-E3 cascade and thus the UPS upstream, is believed to be a powerful alternative for developing anti-cancer agents.

### Methods & Materials

### General Fmoc SPPS Strategy

Synthetic Ub proteins were synthesized by solid phase peptide chemistry following the procedures by E1 Oualid et al. Angew. Chem. Int. Ed Engl. 2010, volume 49, pp 10149.

### Synthesis Ub(1-75)-ΔAla

Method A:

50 μmol Fmoc-[Ub(1-75)]-OH was dissolved in 15 mL DCM and treated overnight with 5 eq pyBOP (0.25 mmol, 130 mg), 10 eq DiPEA (0.5 mmol, 87 μL) and 5 eq H-Cys(Bn)-OMe (HCl salt) (0.25 mmol, 65 mg). The organic layer was washed with 1M KHSO₄, dried with Na₂SO₄ and concentrated. The crude product was dissolved in 20 mL DCM and treated overnight with 5 eq DBU (37 μL) followed by another 2 hrs with fresh DBU (5 eq, 37 μL). The organic layer was washed with 1M KHSO₄, dried with Na₂SO₄ and concentrated. Next, the product was totally deprotected by treatment for 3 hrs in 10 mL of TFA/H₂O/iPr₃SiH/PhOH (90/5/2.5/2.5; v/v/v/v). The crude product was precipitated in 90 mL cold diethyl ether/n-pentane 3/1 v/v and pelleted at 1500 rpm for 10 min; this was repeated 3× using diethyl ether to wash the precipitated product. The product was dissolved in 5 mL DMSO and diluted into 20 mL milliQ containing 5% acetonitrile and 0.05% TFA (buffer A HPLC). The obtained mixture was purified by RP-HPLC using 2 mobile phases: A=0.05% TFA and 5% acetonitrile in milliQ and B: 0.05% TFA and 5% milliQ in CH₃CN. Waters C18 XBridge 5 μM, 130 Å (30×150 mm); flowrate: 30 mL/min. Gradient: 25→75% B over 15 min. Pure fractions were pooled and lyophilized. Yield Ub(1-75)-Cys(Bn)-OMe: 135 mg, 15.5 μma 31%. ES MS+ (amu) calcd: 8715, found 8714. Next, 65 mg (7.5 μmop Ub(1-75)-Cys(Bn)-OMe was dissolved in 1 mL DMSO and diluted into 40 mL 50 mM sodium phosphate pH 8. A solution of MSH (10 eq, 16 mg) in 1 mL DMF was added to the reaction mixture and after stirring for 2 hrs, LC-MS analysis showed complete elimination of the Cys(Bn) into the ΔAla moiety. The reaction had turn cloudy and centrifugation (30 min, 4000 rpm) allowed removal of any precipitated material; LC-MS analysis of this precipitate (dissolved in DMSO) showed that this was not the product. The clear solution containing the product was brought to pH 10 with 10N NaOH. After 90 min, LC-MS analysis showed complete hydrolysis of the methyl ester. RP-HPLC purification as described above, gave the desired Ub(1-75)-ΔAla in good yield. ES MS+ (amu) calcd: 8577, found 8577.

Method B:

Ub G76C (215 mg; 25.0 μmop was dissolved in 2 mL of DMSO and added slowly to MilliQ (75 mL). This was diluted with 100 mM sodium phosphate pH8.0 to a final volume of 150 mL (50 mM NaP pH 8). Next, 2,5-dibromohexandiamide (75.4 mg; 250 μmop was added. The reaction mixture was incubated at 37° C. overnight and spun down to remove the insoluble dibromide. RP-HPLC purification as described above, gave the desired Ub(1-75)-ΔAla (96 mg, 11.2 umol, 45%). ES MS+ (amu) calcd: 8577, found 8577 (FIG. 3 and FIG. 4).

### Synthesis Cy5-Ub(1-75)-ΔAla

200 μmol Cy5-[Ub(1-75)]-OH was dissolved in 25 mL DCM and treated overnight with 5 eq pyBOP (1.00 mmol, 520 mg), 10 eq DiPEA (2.00 mmol, 360 μL) and 5 eq H-Cys(Bn)-OMe (HCl salt) (1.00 mmol, 260 mg). The organic layer was washed with 1M KHSO₄, dried with Na₂SO₄ and concentrated. Next, the product was totally deprotected by treatment for 3 hrs in 20 mL of TFA/H₂O/iPr₃SiH/PhOH (90/5/2.5/2.5; v/v/v/v). The crude product was precipitated in 180 mL cold diethyl ether/n-pentane 3/1 v/v and pelleted at 1500 rpm for 10 min; this was repeated 3× using diethyl ether to wash the precipitated product. The product was dissolved in 20 mL DMSO and diluted into 80 mL milliQ containing 5% acetonitrile and 0.05% TFA (buffer A HPLC). The obtained mixture was purified by RP-HPLC as described for Ub(1-75)-ΔAla. Yield Cy5-Ub(1-75)-Cys(Bn)-OMe: 520 mg, 58 μmol, 35%. Next, 160 mg (18.2 Cy5-Ub(1-75)-Cys(Bn)-OMe was dissolved in 1 mL DMSO and diluted into 115 mL milliQ. This solution was buffered to 50 mM sodium phosphate with a 0.4M sodium phosphate stock of pH 6.8; the pH was adjusted with 10N NaOH to pH 8. A solution of MSH (10 eq, 0.18 mmol, 39 mg) in 0.5 mL DMF was added to the reaction mixture and after stirring for 2 hrs, LC-MS analysis showed complete transformation of the Cys(Bn) group into the expected ΔAla moiety. The reaction had turn cloudy and centrifugation (30 min, 4000 rpm) allowed removal of precipitated material; LC-MS analysis of this precipitate (dissolved in DMSO) showed that this contained no product. The clear solution with the product was brought to pH 10 with 10N NaOH. After 90 min, LC-MS analysis showed complete hydrolysis of the methyl ester. RP-HPLC purification as described above, gave the desired Cy5-Ub(1-75)-ΔAla in 30% yield: ES MS+ (amu) calcd: 9042, found 9041 (FIG. 5).

### Biotin-PEG-Ub(1-75)-ΔAla

20 μmol of fully buffered Biotin-PEG-[Ub(1-75)]-OH was dissolved in 5 mL DCM and treated overnight with 5 eq pyBOP (0.1 mmol, 52 mg), 10 eq DiPEA (0.2 mmol, 36 μL) and 5 eq H-Cys(Bn)-OMe (HCl salt) (0.1 mmol, 26 mg). The organic layer was washed with 1M KHSO₄, dried with Na₂SO₄ and concentrated. Next, the product was totally deprotected by treatment for 3 hrs in 2 mL of TFA/H₂O/iPr₃SiH/PhOH (90/5/2.5/2.5; v/v/v/v). The crude product was precipitated in cold diethyl ether/n-pentane 3/1 v/v and pelleted at 1500 rpm for 10 min; this was repeated 3× using diethyl ether to wash the precipitated product. The product was dissolved in 2 mL DMSO and diluted into 15 mL milliQ containing 5% acetonitrile and 0.05% TFA (buffer A HPLC). The obtained mixture was purified by RP-HPLC as described for Ub(1-75)-ΔAla. Yield Biotin-PEG-Ub(1-75)-Cys(Bn)-OMe: 17 mg, 1.9 μmol, 10%. ES MS+ (amu) calcd: 9086, found 9085. Next, 4.8 mg (0.53 μmol) Biotin-PEG-Ub(1-75)-Cys(Bn)-OMe was dissolved in 0.4 mL DMSO and diluted into 10 mL 50 mM sodium phosphate pH 8. A solution of MSH (10 eq, 1.2 mg) in 100 μL DMF was added to the reaction mixture and after stirring for 2 hrs, LC-MS analysis showed complete transformation of the Cys(Bn) group into a ΔAla moiety. The reaction had turn cloudy and centrifugation (30 min, 4000 rpm) allowed removal of precipitated material; LC-MS analysis of this precipitate (dissolved in DMSO) showed that this contained no product. The clear solution with the product was brought to pH 10 with 10N NaOH. After 90 min, LC-MS analysis showed complete hydrolysis of the methyl ester. RP-HPLC purification as described above, gave the desired Biotin-PEG-Ub(1-75)-Cys(Bn)-OMe (2.5 mg; 0.29 μmol) in 55% yield. ES MS+ (amu) calcd: 8948, found 8948 (FIG. 6 and FIG. 7).

### SPPS Nedd8 G76C

SPPS of Nedd8 G76C was performed on a Syro II MultiSyntech Automated Peptide synthesizer using standard 9-fluorenylmethoxycarbonyl (Fmoc) based solid phase peptide chemistry at 100 μmol scale, a 4-fold excess of amino acids and pyBOP and a 8-fold excess of DiPEA relative to Fmoc-L-Cys(Trt)-PEG-PS (0.20 mmol/g, Applied Biosystems®) resin. All amino acids were double coupled except for the dipeptide building blocks. Dipeptides Fmoc-L-Leu-L-Thr(ΨMe,Mepro)-OH, Fmoc-L-Tyr-L-Ser(ΨMe,Mepro)-OH and Fmoc-L-Gly-L-Ser(ΨMe,Mepro)-OH were single coupled for 1 hour. Fmoc removal was performed with 20% piperidine/NMP for 2×3 min and 1×8 min.

Next, the resin bound Nedd8 G76C was treated with TFA/H₂O/iPr₃SiH/PhOH (90/5/2.5/2.5; v/v/v/v) for 3 h. The crude product was precipitated in cold diethyl ether/n-pentane 3/1 v/v and pelleted at 1500 rpm for 10 min; this was repeated 3× using diethyl ether to wash the precipitated product. The pellet was dissolved in a mixture of H₂O/CH₃CN/HOAc (65/25/10 v/v/v) and finally lyophilized. The product was dissolved in 2 mL DMSO and diluted into 15 mL milliQ containing 5% acetonitrile and 0.05% TFA (buffer A HPLC). The obtained mixture was purified by RP-HPLC as described for Ub-(1-75)-ΔAla. ES MS+ (amu) calcd: 8606, found 8604. The pure fractions were combined, ACN was concentrated in vacuo and the remaining solution was diluted to a final volume of 100 mL in 50 mM sodium phosphate pH 8. Next, 2,5-dibromohexandiamide (500 mg, 1.66 mmol) was added and the reaction mixture incubated overnight at 37° C. overnight. Precipitated material derived from the dibromide was removed and RP-HPLC purification as described above, followed by SE purification gave the desired Nedd8-(1-75)-ΔAla (8 mg, 0.9 μmol, 1%). ES MS+ (amu) calcd: 8572, found 8572.

### Protein Expression and Purification

Uba1 was expressed in E. coli BL21 (DE3) from a pET3a vector with an N-terminal His-tag in autoinduction³ media at 37° C., and switched to 18° C. after 2-3 hours and allowed to grow an additional 12 h. His-tag purification was performed using TALON beads (Clontech) in Buffer A containing 50 mM TRIS (pH 8.0), 150 mM NaCl and 0.25 mM TCEP, washing with 5 mM imidazole (pH 8.0) and eluting with 500 mM imidazole (pH 8.0). Further purification was performed by anion exchange using a Resource Q column (GE Healthcare) using a gradient to 1M NaCl in Buffer A, which was followed by a Superdex 200 column (GE Healthcare).

The untagged Ub G76C mutant was expressed in E. coli BL21 (DE3) cells using autoinduction at 37° C., switched to 18° C. after 2-3 hours and allowed to grow an additional 12 h. Cells were harvested by centrifugation (4000 rpm, 4° C.), resuspended in lysis buffer, lysed by sonication on ice, and centrifuged for 30 min at 20 000 g at 4° C. to remove all cell debris. The soluble lysate was then treated with 2.5 mM beta-mercaptoethanol for 10 min before being heated to 85° C. to precipitate contaminating proteins which were removed by centrifugation (20 000 rpm, 20 min 4° C.). The pH of the cleared lysate (about 90% purity) was adjusted to pH 4.7 using 1M NH₄OAc before being loaded onto a cation exchange column (40S Workbeads, BioWorks). Next, the peptide was purified by using a MonoS column and 0-1 M NaCl gradient in 50 mM NaOAc pH 4.5. Fractions with product were pooled and further purified by prep-HPLC using 2 mobile phases: A=0.05% TFA in milliQ and B: 0.05% TFA in CH₃CN. Prep-HPLC program: Waters C18 XBridge 5 μM, 130 Å (30×150 mm); flowrate: 30 mL/min. Gradient: 0-6 min: 5→25% B; 6-21 min: 25→75% B; 21-23 min: 75→95% B. The purified Ub G76C was lyophilized and used as a precursor for the elimination reaction with the dibromide.

Full length constructs of UBE2N, UBE2V2, and UBE2D3, and NEDD4 residues 521-900, and cIAP residues 363-614 were expressed from pGEX6P-1 constructs in Rosetta 2 (DE3) pLacI cells (Novagen). TRAF6 residues 50-211 were expressed similarly from the pOPIN-K vector. ¹⁵N-labeled UBE2N was expressed in M9 minimal medium supplemented with ¹⁵N—NH₄Cl. Transformed cells were cultured at 37° C. to an O.D. of 0.6-0.8 and induced with 0.2 mM IPTG at 18° C. for 16 hours. Cells were lysed by sonication in a buffer containing 25 mM Tris (pH 8.5), 200 mM NaCl, and 4 mM DTT. The clarified lysate was bound to glutathione resin, washed with additional lysis buffer, and the resin was incubated with GST-tagged 3C protease overnight at 4° C. for cleavage of the GST fusion tag, with the exception of NEDD4 and cIAP which were eluted to retain the GST tag. Following cleavage, the released protein was washed through the resin, concentrated, and further purified by size exclusion chromatography on a Superdex 75 column (GE Healthcare) equilibrated in either 20 mM Tris (pH 7.4), 100 mM NaCl, 1 mM DTT for reactions, or 25 mM sodium phosphate (pH 7.0), 150 mM NaCl for NMR spectroscopy. Mouse His-UBE1 was expressed from the pet-24 vector as above and purified by conventional Ni-NTA methods followed by anion exchange on a Resource Q column and size exclusion chromatography on a Superdex 200 column (GE Healthcare). Pure fractions of all proteins were concentrated and flash frozen for storage at −80° C.

### E1 Labeling Assay

Uba1 or Uba6 (1 μM) is incubated with probe (30 μM) in buffer containing 50 mM HEPES and 100 mM NaCl (pH 8.0) containing 10 mM MgCl₂ and 10 mM ATP at 37° C. for 30 min. The reaction was quenched by the addition of reducing sample buffer and heating (90° C. for 10 min). Samples were separated by SDS-PAGE and then stained with Silver stain for analysis.

### E2 Labeling Assay

E2 enzyme (2.5 μM) and Uba1 (0.63 μM) were incubated with probe (12.5 μM) in buffer containing 50 mM HEPES and 100 mM NaCl (pH 7.5) containing 5 mM MgCl₂ and 2 mM ATP at 37° C. for 30 min. The reaction was quenched by the addition of reducing sample buffer and heating (90° C. for 10 min). Samples were separated by SDS-PAGE and then stained with Silver stain for analysis.

### E2 Labeling Assay—Comparison Probe Ub(1-75)-ΔAla and Native Ub76

The labeling of UbcH7 with probe Ub(1-75)-ΔAla was compared to native Ub76. UbcH7 (2.5 μM) and Uba1 (0.3 or 1.25 μM) were incubated with probe (25 μM) or Ub76 (25 μM) in buffer containing 50 mM HEPES and 100 mM NaCl (pH 7.5) containing 5 mM MgCl₂ and 2 mM ATP at 37° C. for 30 min. The reaction was quenched by the addition of reducing or non-reducing sample buffer. The samples with reducing sample buffer were heated (90° C. for 10 min). Samples were separated by SDS-PAGE and then stained with Silver stain for analysis.

### E2 Labeling Assay—E2^{scan} Kit Panel 34 Enzymes

The E2^{scan} plate (cat. nr. 67-0005-001, Ubiquigent, Dundee, UK) was thawed on ice prior to use. The plate contains a panel of 34 E2 enzymes in duplicate. For this assay this duplicate was used for a non-ATP control. Final concentration of enzymes: E1/E2/probe=0.63/2.5/12.5 μM in a reaction buffer containing 50 mM HEPES pH 7.5, 5 mM MgCl₂ and 2.5 mM DTT.

To 10 μl of E2 on the plate (5 μM), 5 μl of E1/probe mix (2.5 μM/50 μM) was added, followed by 5 μl of 8 mM ATP or 5 ul buffer (control). The plate was incubated for 2 hrs at 30° C. and the reactions were quenched by the addition of reducing (BME) sample buffer and heating (90° C., 10 min). Samples were separated by SDS-PAGE and then stained with Silver stain for analysis. Note: the ubiquitin loading activity of the enzymes with native Ub ranges from 0-70%

### E3 Labeling Assay

Nedd4L (2.5 μM) and UBE2D (0.5 μM) UBE1 (0.25 μM) were incubated with Cy5-probe (50 μM) in 50 mM HEPES pH 7.5, 100 mM NaCl, 5 mM MgCl₂ and 2 mM ATP at 30° C. for 2 h. The reaction was quenched by the addition of reducing sample buffer and heating (90° C. for 10 min). Samples were analyzed by SDS-PAGE and visualized by fluorescence scanning (λex=625 nm; λem=680 nm).

### E3 Panel Labeling

Nine HECT E3 enzymes (HECT domain of human Nedd4, Nedd4L, ITCH, UBE3C, WWP1, WWP2, HACE1, WW3+HECT domain of Smurf2 (human) and Rsp5 (S. cerevisia); 2.5 μM) were incubated with UBE2D (0.5 μM) UBE1 (0.25 μM) and probe (50 μM) in 50 mM HEPES pH 7.0, 100 mM NaCl, 5 mM MgCl₂ and 2 mM ATP at 30° C. for 1 h. Samples were analyzed by SDS-PAGE and visualized by silver stain or western blot (blotted against Ub).

### Nedd8-(1-75)-ΔAla Labeling Assay

UBA3/NAE1 (2 μM) was incubated with Nedd8-(1-75)-ΔAla (40 μM) or Nedd8 (40 μM) in 50 mM Tris pH 7.5, 300 mM NaCl, 10 mM MgCl₂ and 5 mM ATP at 30° C. for 60 min. The reaction was quenched by the addition of reducing or non-reducing sample buffer. The samples with reducing sample buffer were heated (90° C. for 10 min). Samples were separated by Samples were analyzed by SDS-PAGE and visualized by silver stain.

UBE2M wt (2 μM) was incubated with UBA3/NAE1 (0.5 μM) and Nedd8-(1-75)-ΔAla probe (20 μM) in buffer containing 50 mM Tris pH 7.5, 300 mM NaCl, 10 mM MgCl₂ and 5 mM ATP at 30° C. for 60 min. The reaction was quenched by the addition of reducing sample buffer and heating (90° C. for 10 min). Samples were analyzed by SDS-PAGE and visualized by silver stain.

SUMO1(1-96)-ΔAla, SUMO2(1-92)-ΔAla and SUMO3(1-91)-ΔAla Labeling assay

SAE1/2 (2 μM) was incubated with SUMO1(1-96)-ΔAla, SUMO2(1-92)-ΔAla or SUMO3(1-91)-ΔAla (40 μM) in 50 mM HEPES pH 7.8, 150 mM NaCl, 5 mM MgCl₂ and 5 mM ATP at 37° C. for 60 min. The reaction was quenched by the addition of reducing sample buffer and heated at 90° C. for 10 min. Samples were analyzed by SDS-PAGE and visualized by Coomassie G250.

### Structure Determination

Preparation of thioether-linked ¹⁵N-UBE2N-Ub adduct and UBE2D3-Ub adduct: 25 μM UBE1, 250 μM E2 (¹⁵N-UBE2N or UBE2D3), 1 mM Ub-(1-75)-ΔAla, 5 mM ATP, and 10 mM MgCl₂ were incubated at room temperature for 16 hours. Any remaining thioester linkages were reduced by the addition of 10 mM DTT at the end of the reaction. The ¹⁵N-UBE2N-Ub adduct was purified to ˜95% homogeneity by size exclusion chromatography on a Superdex 75 column (GE Healthcare) equilibrated in 25 mM sodium phosphate (pH 7.0), 150 mM NaCl. The UBE2D3-Ub adduct was subsequently purified to >98% homogeneity by repeated cation exchange chromatography on a 6 mL Resource S column (GE Healthcare) with a 50-500 mM NaCl gradient in 50 mM Tris (pH 8.5) buffer, followed by a final size exclusion chromatography step on a Superdex 75 column (GE Healthcare) equilibrated in 25 mM HEPES, 50 mM NaCl pH 7.

NMR spectroscopy ¹⁵N-UBE2N-Ub adduct: ¹H, ¹⁵N BEST-TROSY experiments on 200 μM ¹⁵N-UBE2N or ¹⁵N-UBE2N-Ub adduct were acquired with optimized pulse sequences on a Bruker Avance2+ 700 MHz spectrometer equipped with a cryogenic triple resonance TC1 probe. Data sets were processed using Topspin (Bruker) and visualized with NMRViewJ (One Moon Scientific). Chemical shift perturbations were calculated using the equation Δδⱼ=[(Δδⱼ¹⁵N/5)²+(Δδⱼ¹H)²]^{1/2}. Perturbation values greater than 0.05 ppm were mapped onto the UBE2N crystal structure (PDB 1J7D), such that the analysis is directly comparable to previous work on the oxyester-linked UBE2N—O˜Ub conjugate.

### Crystallization, Data Collection, and Structure Determination

[UBE2D3]-Ub adduct: The thioether-linked UBE2D3-Ub adduct was crystallized following conditions published for the oxyester-linked form. Purified conjugate was concentrated to 0.9 mM (23.5 mg/mL) and screened against conditions surrounding the published 200 mM tripotassium citrate, 20% PEG 3350 condition in 200 nL sitting drops at a 1:1 protein:reservoir ratio. The resulting crystals were cryoprotected in LV CryoOil (MiTeGen) and vitrified prior to data collection at Diamond Light Source beamline 104-1. Diffraction images were processed using XDS and scaled using AIMLESS. Isolated UBE2D3 and Ub molecules from the oxyester UBE2D3-O˜Ub structure (PDB 3UGB) were used as search models for molecular replacement using PHASER. Iterative model building and refinement were performed using COOT and PHENIX, respectively. The thioether linkage could be built unambiguously into the electron density. Data collection and refinement statistics can be found in Table 1. Structural figures were generated using PyMOL (www.pymol.org).

### In Vitro Ubiquitination Assays

Stability: The stability of thioether-linked UBE2D3-Ub(1-75)-ΔAla adduct was assessed by incubating 5 μM purified thioether conjugate at 37° C. in combination with 2 μM TRAF6 or 2 μM GST-NEDD4. The stability of the UBE2N-Ub(1-75)-ΔAla adduct was assessed similarly, but using the accessory E2 variant UBE2V2 with or without TRAF6. Samples were collected at 0, 1, 3, and 24-hour time points for visualization on Coomassie-stained SDS-PAGE gels. Control reactions containing 0.5 μM UBE1, 5 μM E2, 20 μM Ub, and the relevant activating factors demonstrate the Ub chain formation activity under normal in vitro conditions.

Competitive Inhibition:

Single-turnover ubiquitination assays were established for UBE2N with an initial activation stage containing 10 μM UBE2N, 0.5 μM UBE1, 20 μM Ub, 2.5 mM ATP, and 5 mM MgCl₂ to generate native thioester linked UBE2N˜Ub after 30 min at 37° C. This reaction was then quenched by addition of apyrase to prevent any further formation of UBE2N˜Ub. Pre-activated UBE2N˜Ub was then diluted twofold into a reaction containing 5 μM UBE2V2 alone or in addition to 1 μM GST-cIAP, and samples were collected at 0, 15, and 30-minute timepoints for visualization of diUb product formation on Coomassie-stained SDS-PAGE gels. To test for competitive inhibition using the thioether-linked UBE2N-Ub(1-75)-ΔAla adduct, increasing concentrations (1.25 μM, 2.5 μM, 5 μM, 10 μM, and 20 μM) of purified conjugate were added into reactions containing UBE2N˜Ub, UBE2V2, and GST-cIAP as specified above, and 30-minute timepoints were collected to compare levels of diUb product formation with the control experiment.

### Activity Based Protein Profiling

Cell Lysate Labeling Experiments:

100 μg of HEK293, HeLa, MCF-7 or E1-4 cell lysate was incubated with increasing concentrations of Cy5-Ub-(1-75)-ΔAla, 10 mM MgCl₂, 10 mM ATP and 20 nM UBE1 in labeling buffer (100 mM HEPES, 100 mM NaCl, pH 7.5) at 37° C. for 1 h. The reaction was quenched by the addition of reducing sample buffer. Samples were analyzed by SDS-PAGE and visualized by fluorescence scanning (λₑₓ=625 nm; λₑₘ=680 nm).

Confocal Microscopy:

HeLa cells cultured under standard growth conditions in DMEM (Gibco) supplemented with 10% FCS (Sigma-Aldrich) at 37° C. with 5% CO2 were transfected with GFP or USE1-GFP using Effectine (Qiagen), according to manufacturer's instructions. Cells were seeded on covers slips in a 6 well plate to get 60-80% confluence the next day. After removal of the growth medium the 6 well plate was placed on ice and the cells were washed twice with cold electroporation buffer. Next, 1 mL of a solution of the probe in electroporation buffer (0.4 mg/mL) was added to each of the wells and electroporation was performed on ice using a Biorad GenePulser Xcell with CE and PE module Pulse Generator equipped with a Petri Pulser™ electroporation applicator (BTX) using the following settings: square wave, voltage=75V, pulse length=3 ms, pulse interval=1.5 s, number of pulses=5, cuvette width=2 mm. The electroporation applicator was turned 90 degrees and electroporation was repeated once. The probe solution was replaced by cold electroporation buffer and cells were allowed to recover on ice for 5 min. Following treatment, cells were allowed to recover under standard growth conditions for 1 hour, fixed in 4% Formaldehyde (Merck) in PBS and mounted onto glass slides (Thermo Scientific) using Prolong Gold mounting medium with DAPI (Invitrogen). Images were collected on a Leica SP5 confocal microscope equipped with HyD detectors, using a 63× magnification lens in combination with 7× digital zoom. Image processing was performed using ImageJ64 software, and colocalization was expressed in the form of Mander's overlap coefficients calculated using JACoP.

### Peptide Inhibitors of Ub-E1 Thioester Formation

Peptides VYRFYGG and VYRFYGΔAla of varying concentrations (10, 50, 100, 200, 500, 700, 1000 μM) were incubated with 0.3 μM Uba1 in the presence of 1 mM ATP and 10 mM MgCl₂ in TBS buffer (50 mM Tris, 150 mM NaCl, pH=7.5) for 15 min at room temperature. TAMRA-Ub (1 μM) was added to the reaction and samples were taken at two time points, respectively 15 sec and 15 min. The proteins were separated by SDS-PAGE and the TAMRA-Ub-E1 conjugates were visualized using a fluorescence scanner (λₑₓ=530 nm; λₑₘ=580 nm).

### DESCRIPTION OF THE FIGURES

FIG. 1: In situ activation of Ub(1-75)-ΔAla with ATP provides a mechanism based ABP. Pathway a) describes the covalent trapping of the enzyme, while via pathway b) the probe can be processed further downstream.

FIG. 2: Synthesis of the activity based probe Ub(1-75)-ΔAla with and without a fluorescent label.

FIG. 3: Ub(1-75)-Cys(Bn)OMe. Diode Array chromatogram (left). Deconvulated mass of product peak (right). ESI-Mass [M+H] Expected: 8715, Found 8714

FIG. 4: Ub(1-75)-ΔAla. Diode Array chromatogram (left). Deconvulated mass of product peak (right). ESI-Mass [M+H] Expected: 8577, Found 8577

FIG. 5: Cy5-Ub(1-75)-ΔAla. Diode Array chromatogram (left). Deconvulated mass of product peak (right). ESI-Mass [M+H] Expected: 9042, Found 9041

FIG. 6: Biotin-PEG-Ub(1-75)-Cys(Bn)OMe. Diode Array chromatogram (left). Deconvulated mass of product peak (right). ESI-Mass [M+H] Expected: 9086, Found 9085

FIG. 7: Biotin-PEG-Ub(1-75)-ΔAla. Diode Array chromatogram (left). Deconvulated mass of product peak (right). ESI-Mass [M+H] Expected: 8948, Found 8948

FIG. 8: ATP dependant labeling of the E1 enzymes a) Uba1; b) Uba6

FIG. 9: Reactivity of UbcH7 against native Ub and UbΔAla probe under non- and reducing conditions. Asterisks (*) indicate the modified form of UbcH7. a) Molar ratio E1/E2:0.1/1; b) Molar ratio E1/E2:0.5/1.

FIG. 10: Ub-(1-75)-ΔAla labels 27 E2s specific for Ub transfer but not E2s employing Ub1s. The asterisks indicate the thioether-linked E2-AlaUb adducts. (E2-scan kit, Ubiquigent). Visualized by silver stain.

FIG. 11: Fluorescent scan showing Nedd4L HECT labeling with the Cy5-Ub-(1-75)-ΔAla Asterisks (*) indicate the modified form of Nedd4L.

FIG. 12: Ub(1-75)-ΔAla shows reactivity towards E3 HECT enzymes under ATP dependent conditions. The asterisks indicate the E3-Ub(1-75)-ΔAla thioether-linked adduct. Visualized by silver staining (upper panel) and western blot against Ub (lower panel).

FIG. 13: The thioether-linked UBE2N-AlaUb adduct behaves like the oxyester linkage in solution a) Full ¹H, ¹⁵N HSQC spectra of UBE2N (black) and thioether-linked UBE2N-Ub(1-75)-ΔAla (red). b) Quantified chemical shift perturbations in UBE2N upon activation with Ub for the oxyester (black) and thioether (gray) linkages. Resonances that were either completely exchanged broadened or shifted beyond facile re-assignment are plotted with the maximum perturbation value. c) Changes occurring upon thioether linkage are mapped onto the UBE2N structure (PDB 1J7D). Based upon chemical shift perturbation values in b) and manual inspection of the overlaid spectra, changes that closely resemble those observed for the oxyester linkage could be mapped (primarily localized to Loop 3, Helix 2, Loop 8, and the penultimate C-terminal helix), as well as changes that differ from those observed for the oxyester linkage (region directly surrounding the active site).

FIG. 14: The crystal structure of thioether-linked UBE2D3-AlaUb shows high similarity to the active site conformation of the oxyester conjugate a) Superposition of thioether-linked UBE2D3-AlaUb conjugate (dark gray) with the previously determined structure of the oxyester-linked form (light grey dotted pattern, PDB 3UGB). b) Simulated annealing omit map showing the electron density surrounding the UBE2D3-AlaUb thioether linkage with UbAla76 absent from the model. 2mFo-Fc map (dark grey) is contoured at 1σ, the mFo-Fc map (light grey) is contoured at 3σ. Inlay: flattened diagram illustrating the thioether linkage. c) Overlay of E2 active site residues in the thioether (dark gray) and oxyester (light grey dotted pattern) structures.

FIG. 15: Thioether-linked adducts are inert. a) An SDS PAGE of a reaction time course demonstrates the stability of the thioether-linked UBE2D3-AlaUb adduct when incubated in combination with the RING E3 ligase TRAF6 or the HECT E3 ligase NEDD4. Control reactions show the activities of these E3 ligases with wildtype components. b) Stability of the thioether-linked UBE2N-AlaUb adduct when incubated with the accessory E2-variant UBE2V2 alone or in combination with the RING E3 ligase TRAF6. Control reactions show the activities of these enzymes with wildtype components.

FIG. 16: Thioether-linked UBE2N-AlaUb adduct can compete with downstream ubiquitination enzymes. Single-turnover ubiquitination assay monitoring the formation of diUb from thioester-linked UBE2N˜Ub. Titration of the stable thioether-linked UBE2N-AlaUb into the reaction results in diminished diUb production.

FIG. 17: In-gel fluorescence imaging of activity profiling in cell lysates a) Labeling of HEK293, HeLa, MCF-7 and EL-4 cell lysates with increasing amounts of Cy5-Ub(1-75)-ΔAla. The probe only control is shown for the highest concentration; aggregation of probe on SDS-PAGE is visible at high probe concentration (Fluorescent scan); b) Labeling of a panel of tumor cell lines.

FIG. 18: Visualizing the activity of Ub conjugating enzymes in cells using a fluorescent Ub(1-75)-ΔAla probe a) Cy5-Ub(1-75)-ΔAla probe or TAMRA-Ub was introduced into HeLa cells by electroporation. Following 1 hr recovery period, cells were fixed and visualized by confocal microscopy. Overlays with nuclear DAPI staining are shown; scale bars=10 μm. b) Cells harboring Cy5-Ub(1-75)-ΔAla in various mitotic phases. Overlays with DAPI (blue) and corresponding transmission images are shown; scale bars=c) Cells ectopically expressing either free GFP or USE1-GFP treated with Cy5-Ub(1-75)-ΔAla as in a). Overlays with DAPI are shown. Pixel intensity plots (right panels) for the corresponding pairs of Cy5 (y-axis) and GFP material (x-axis) are shown. Mander's overlap coefficients are represented as a fraction of Cy5-Ub(1-75)ΔAla overlapping free GFP versus USE1-GFP; n=2, error bar=SD, *** p<0.001.

FIG. 19: Nedd8(1-75)-ΔAla shows covalent bond formation with the E1 Uba3 and E2 UBE2M (visualized by silver stain). The asterisks indicate the labeled enzyme adducts.

FIG. 20: SUMO1, SUMO2 and SUMO3 based ΔAla probes show covalent bond formation with SUMO E1 enzyme which functions as a heterodimer. The SAE2 harbours the active cysteine site while SUMO transfer to the E2 conjugating enzyme requires both of the SAE subunits.

FIG. 21: Fluorescent scanning analysis of Uba1 loading with TAMRA-Ub after pre-incubation with the peptide a) VYRFYGG or b) VYRFYG-ΔAla. Inhibition of TAMRA-Ub-E1 thioester formation was measured after 15 s incubation with peptide (top gel) and 15 min of incubation (bottom gel).

FIG. 22: Inhibitory activity of the two peptides (after 15 min incubation) as quantified from the fluorescence gel scanning results in FIG. 21. As can be seen, replacement of the C-terminal Gly of VYRFYGG by an ΔAla leads to a peptide (VYRFYGΔAla) with a higher inhibitory potency against Uba1 activity.

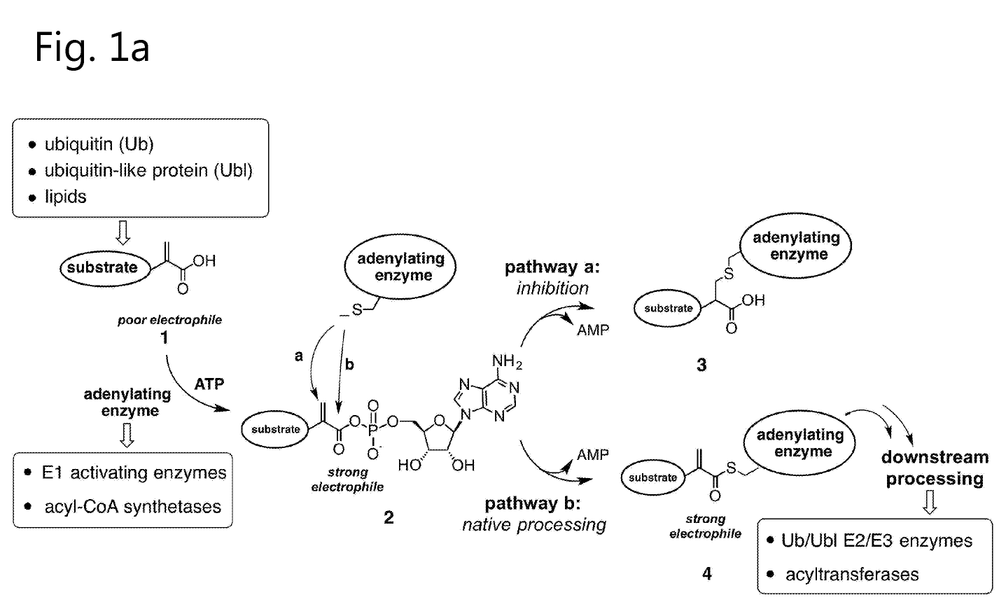

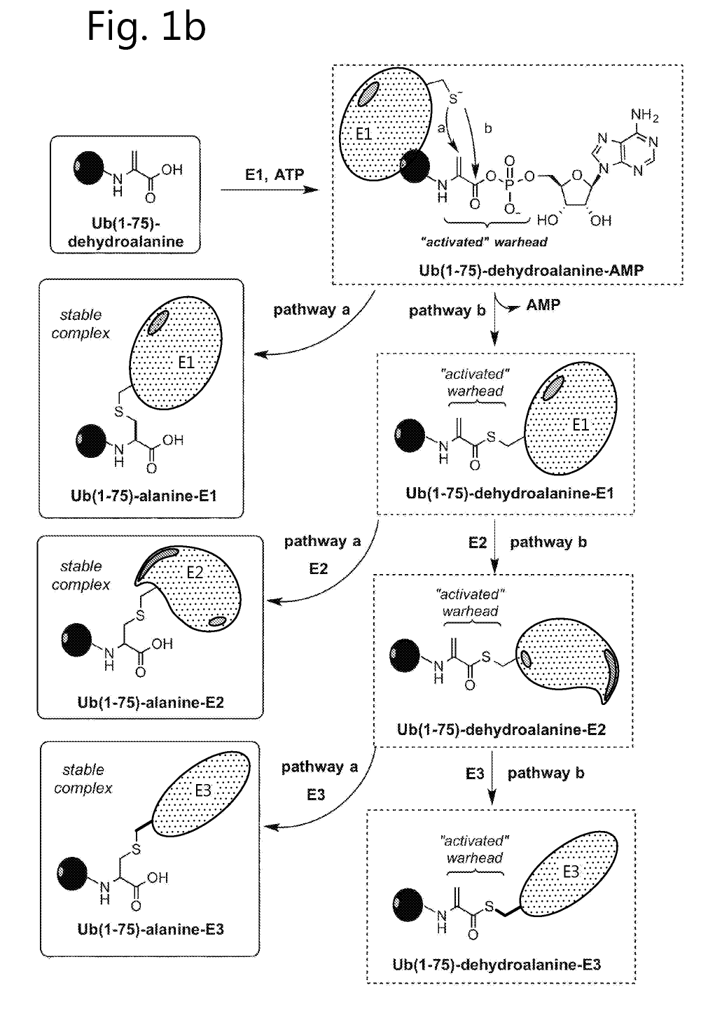

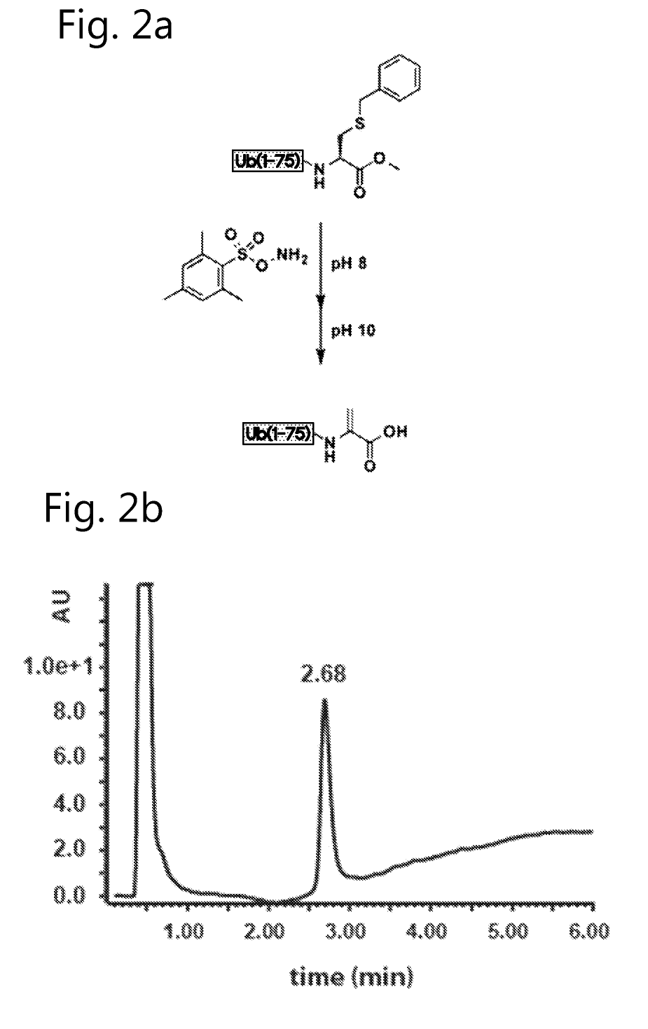

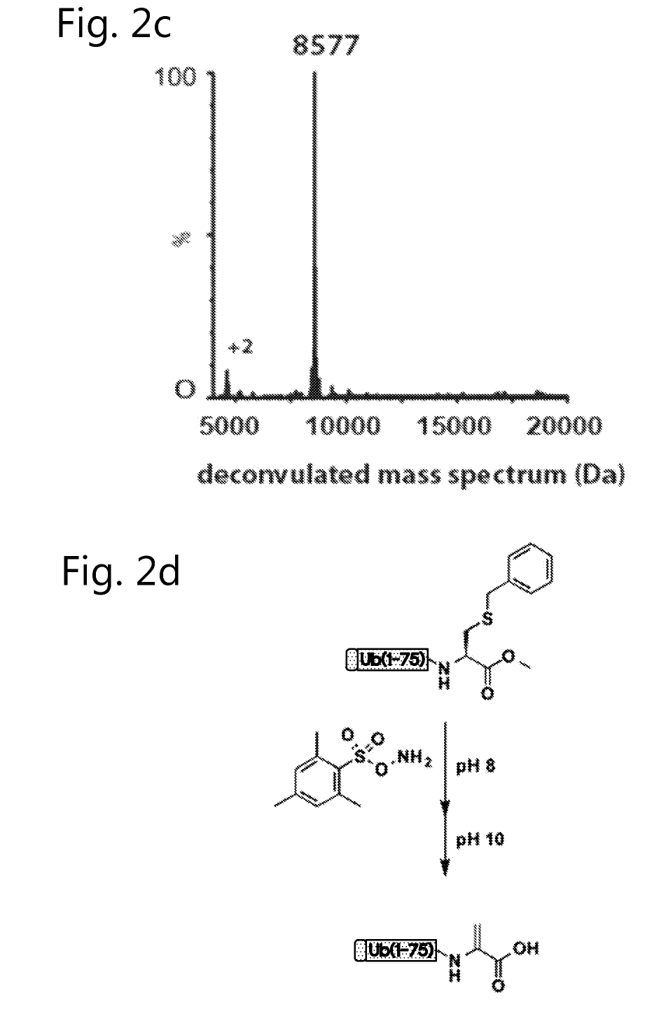

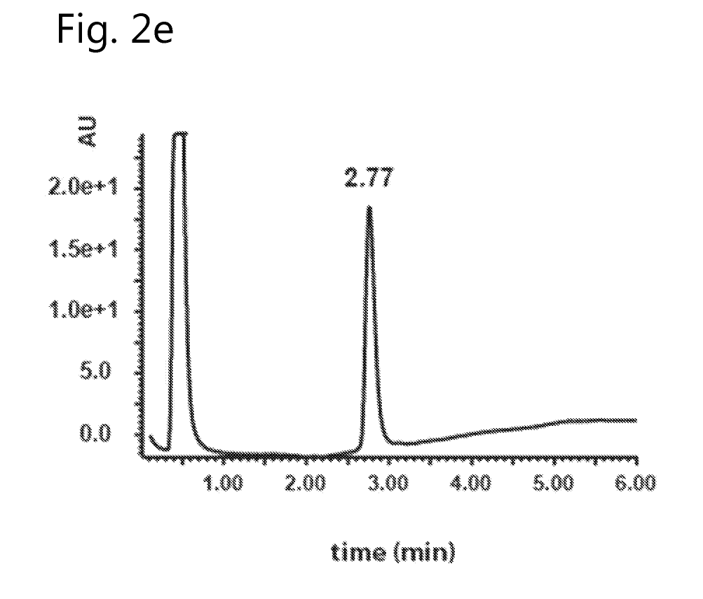

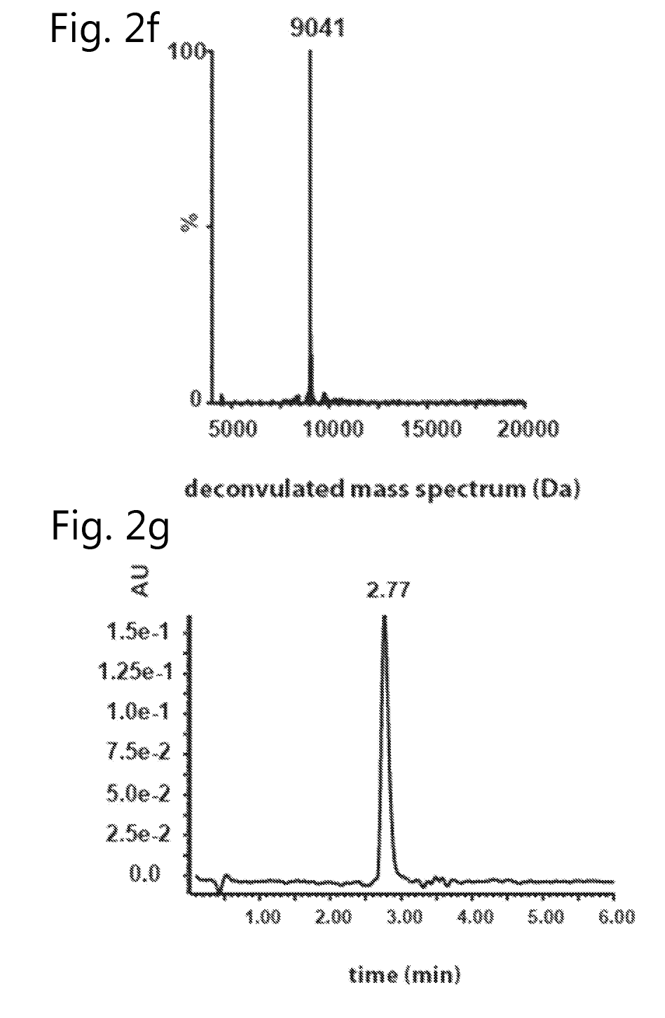

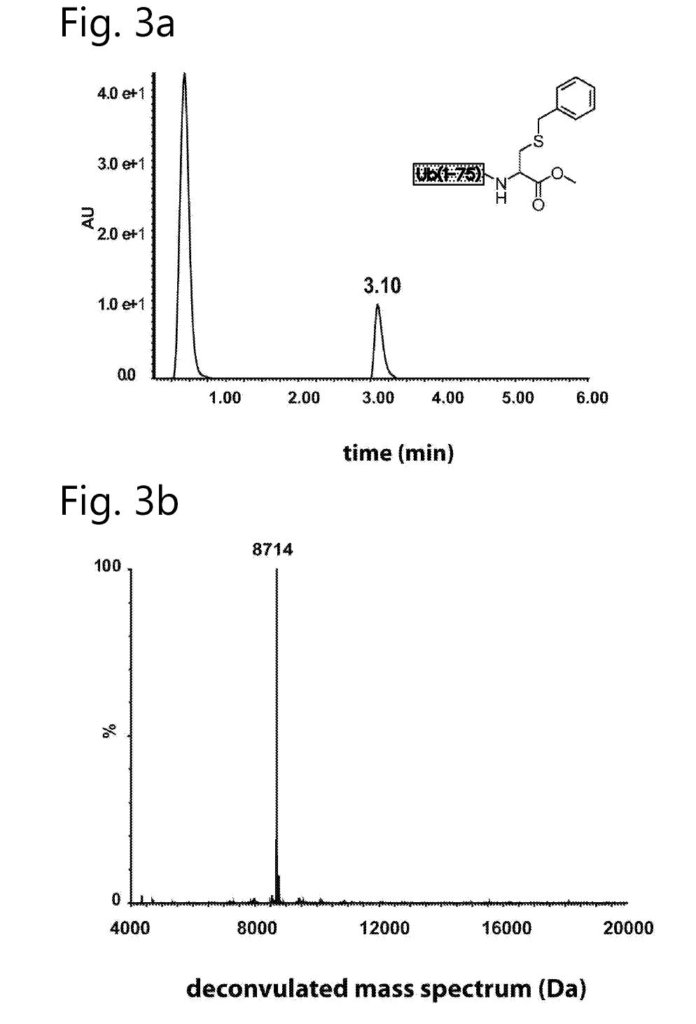

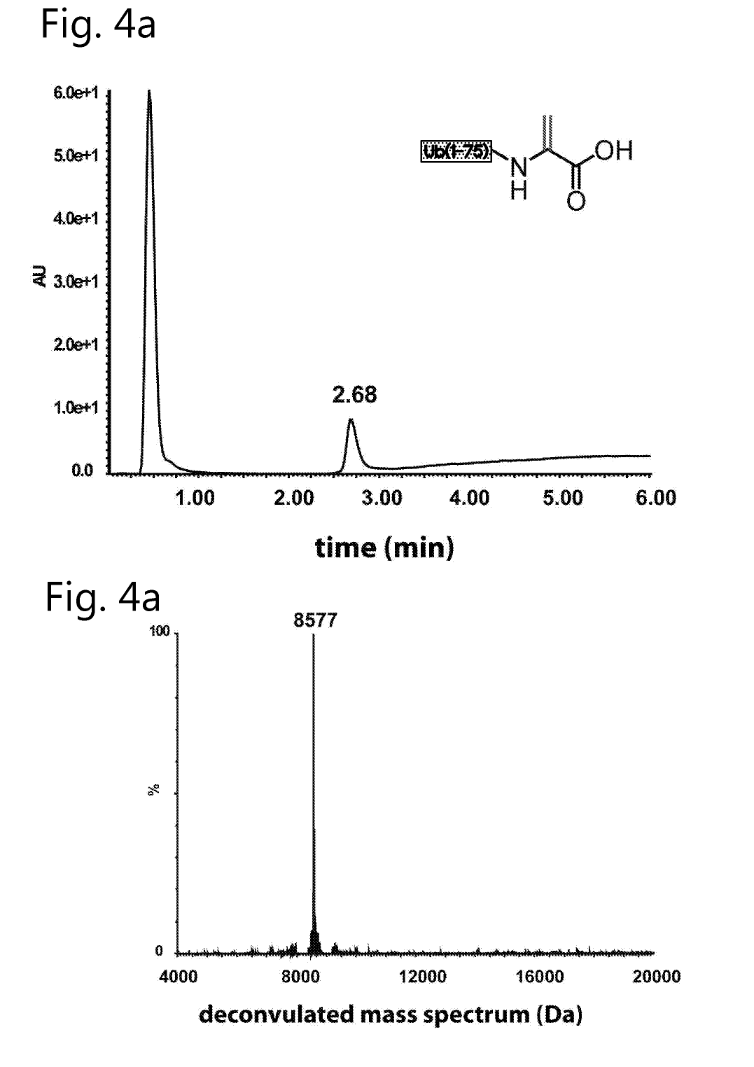

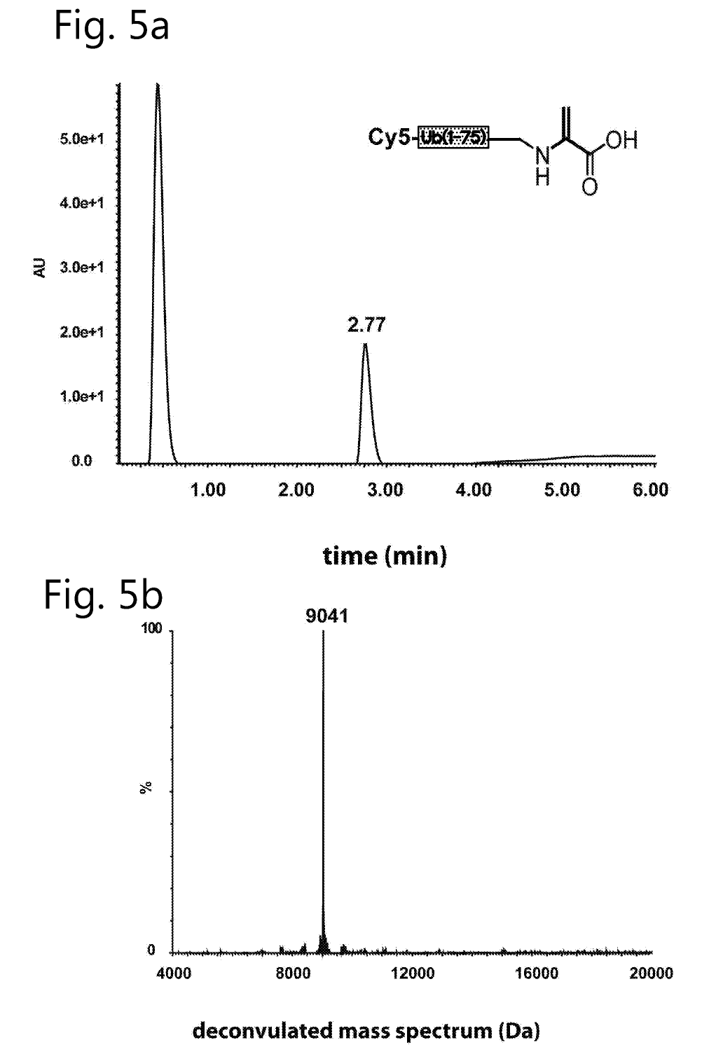

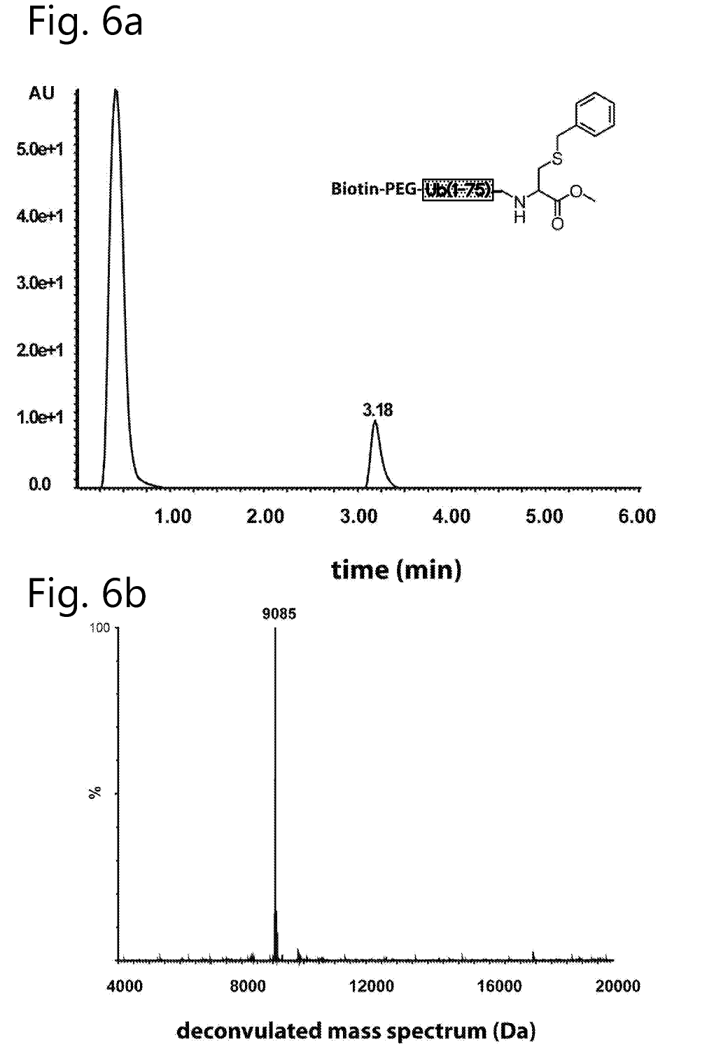

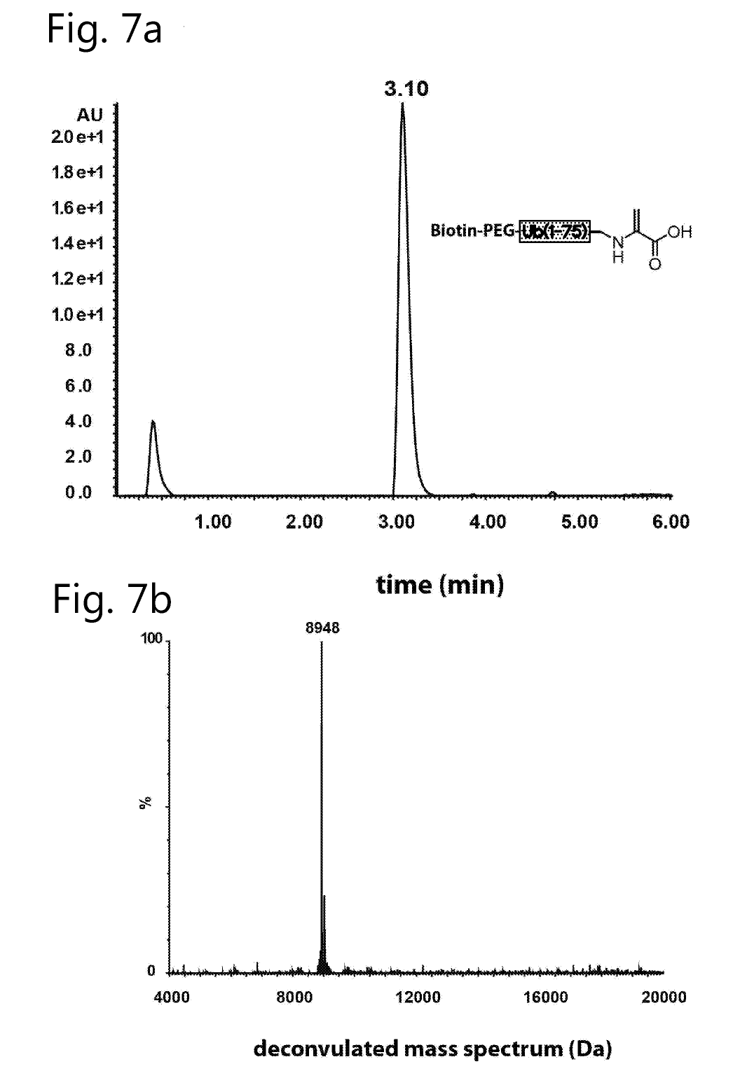

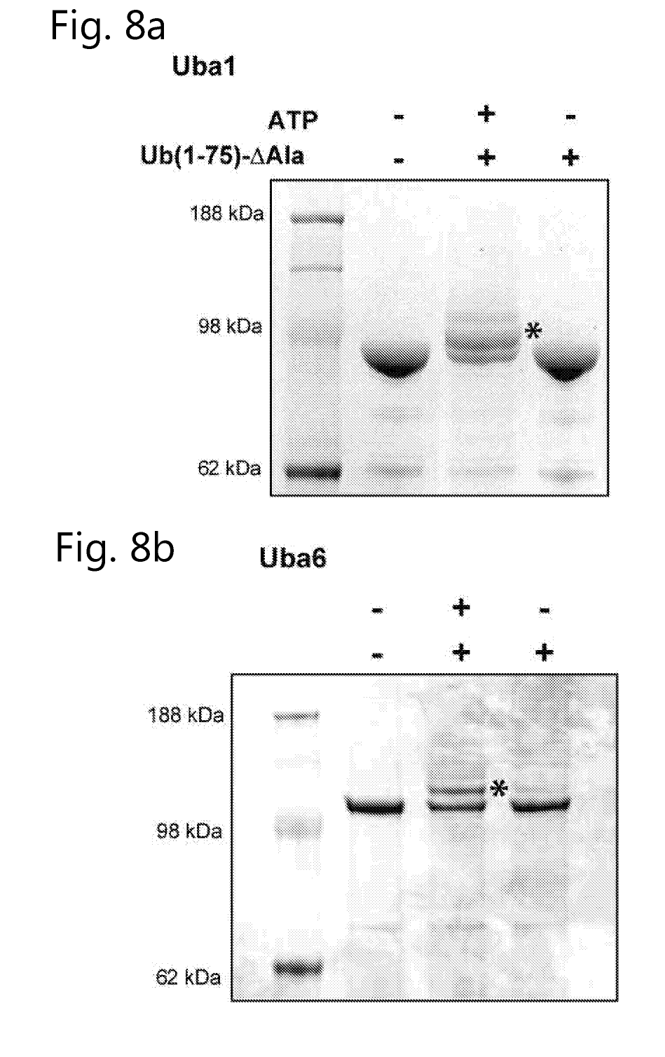

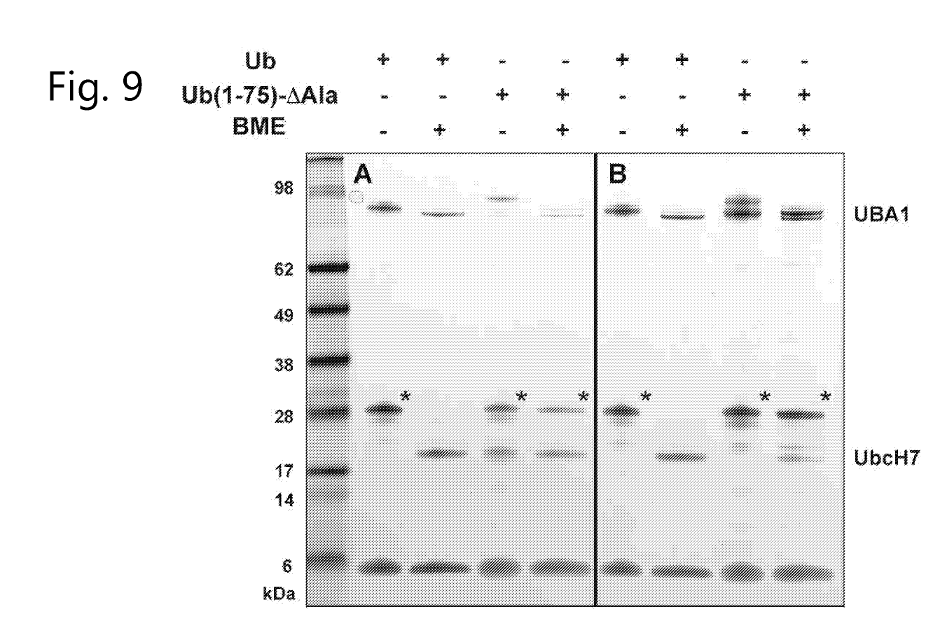

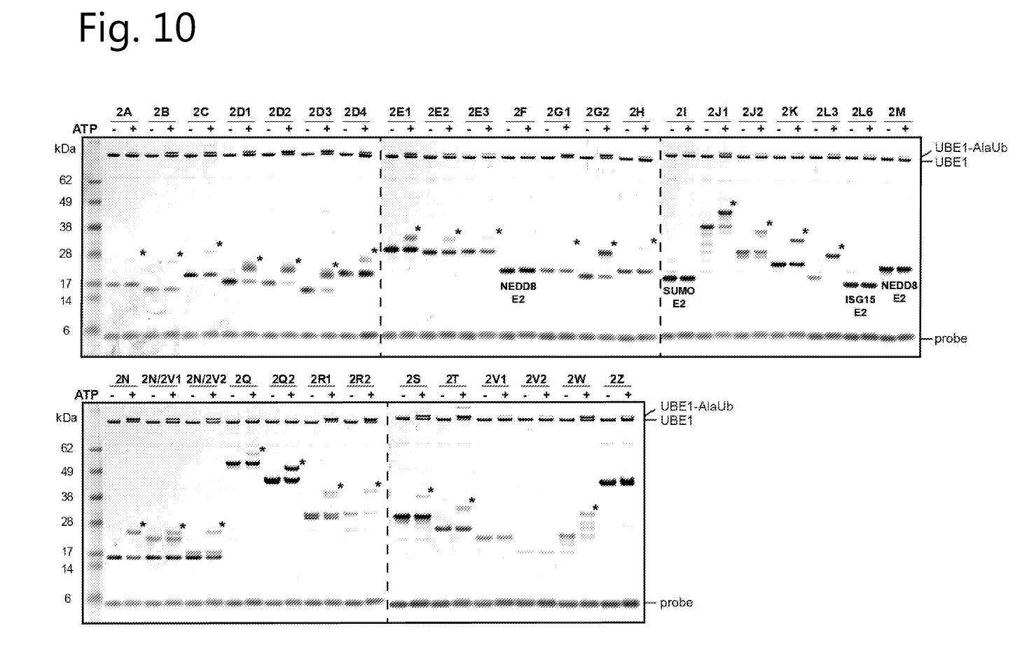

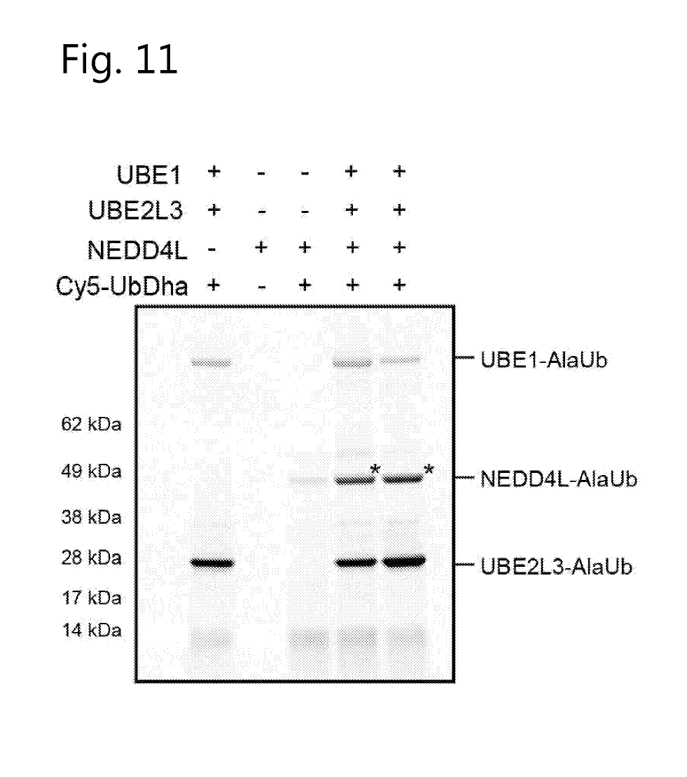

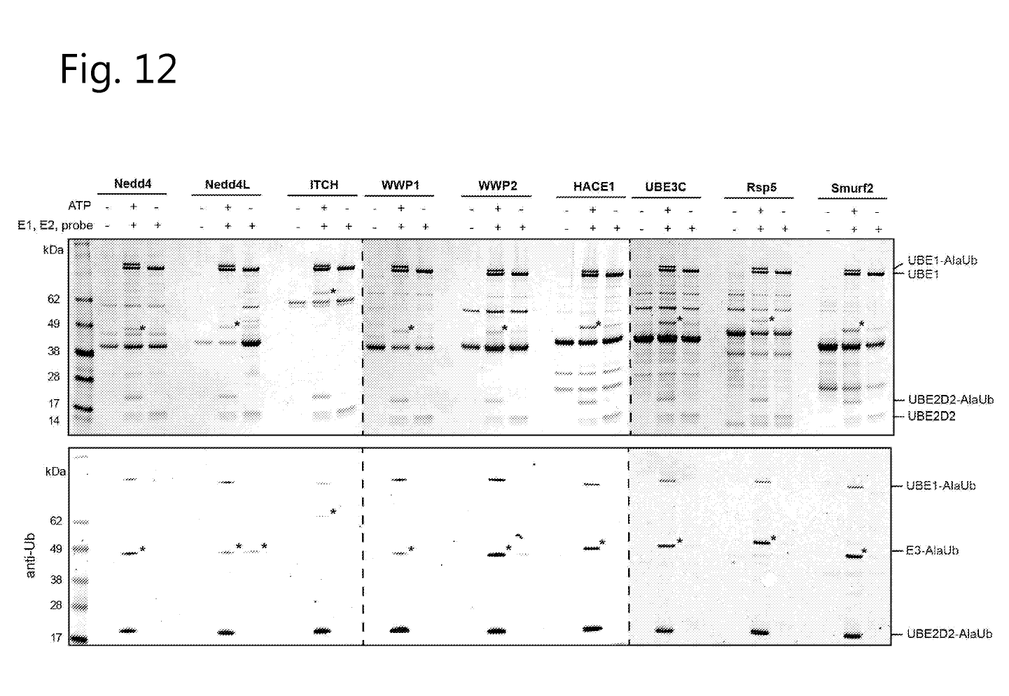

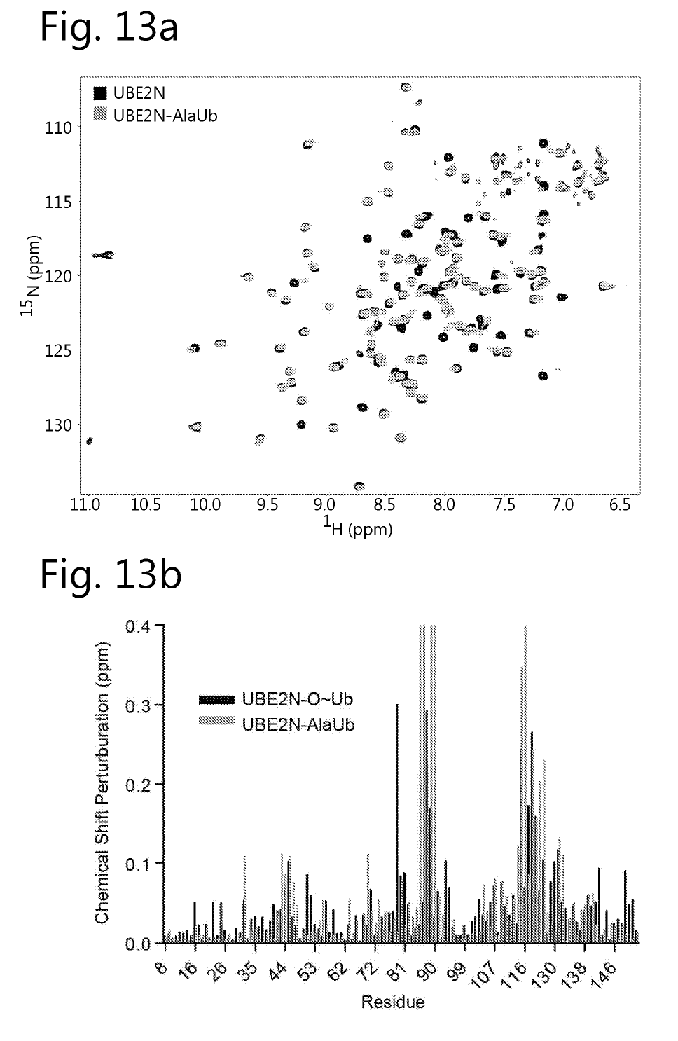

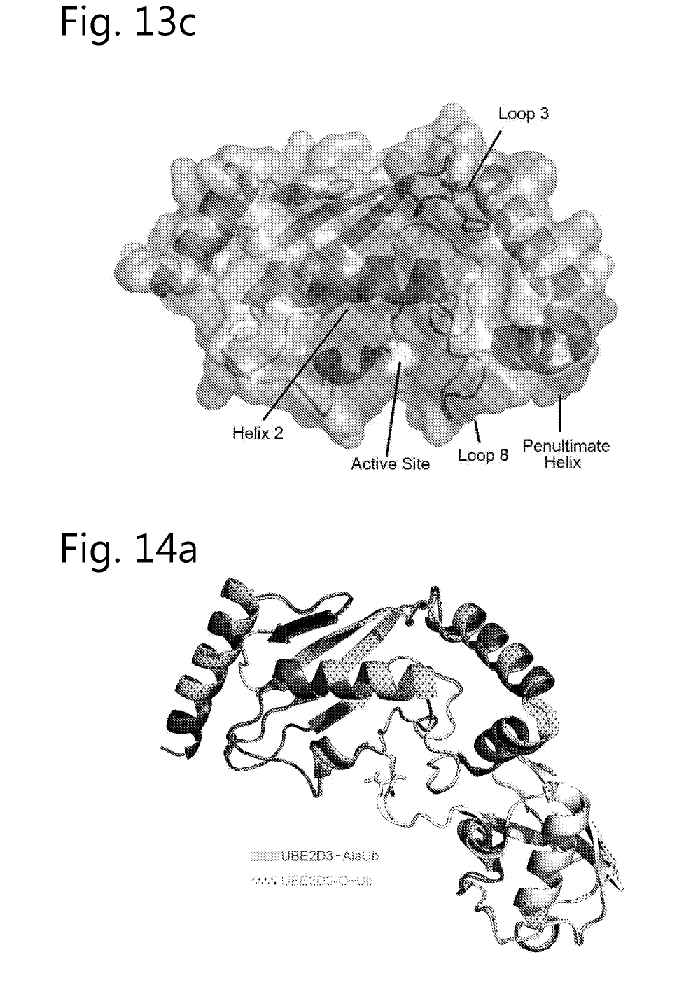

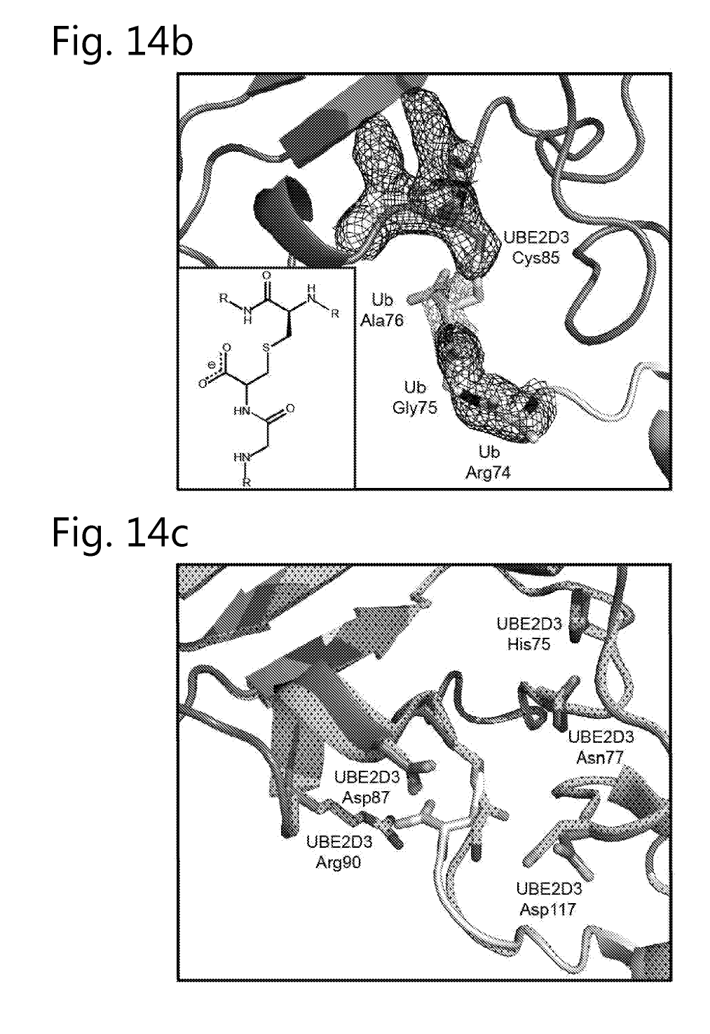

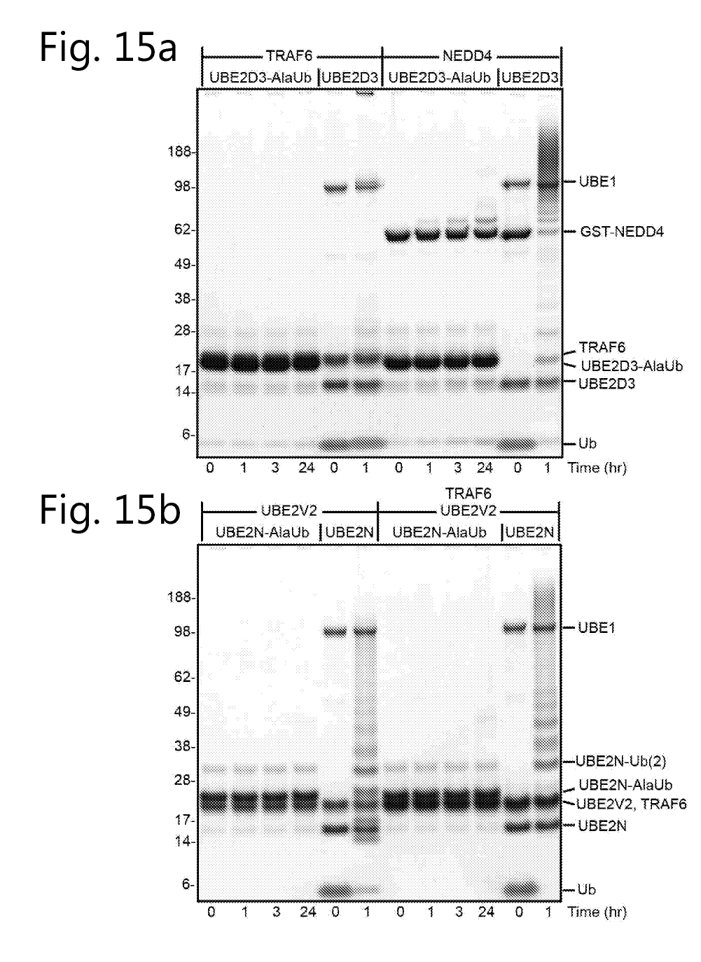

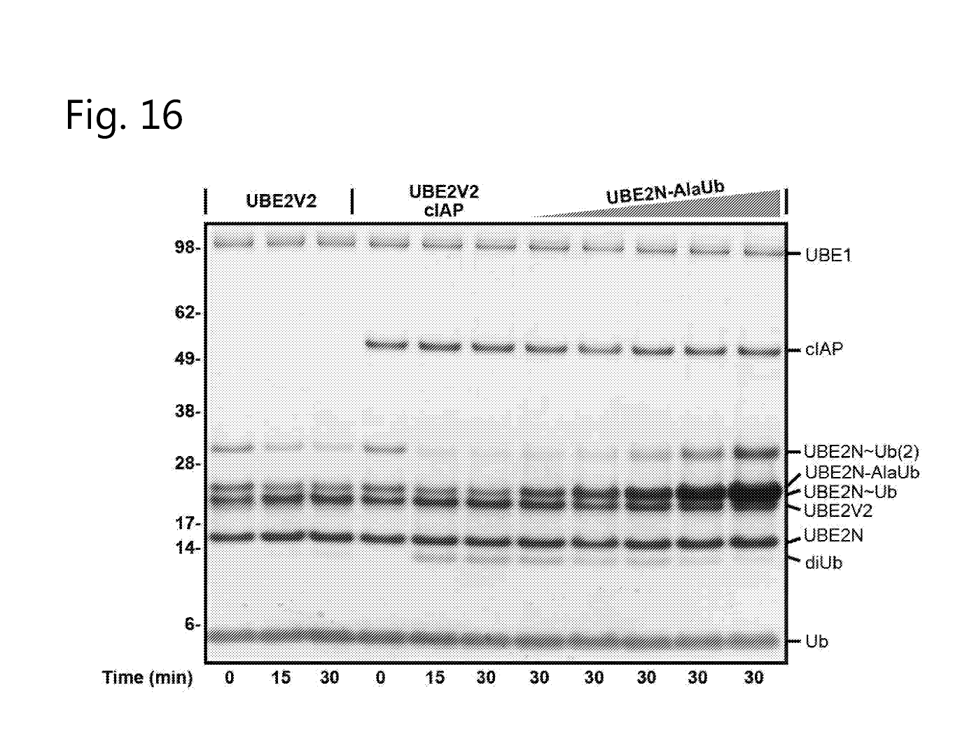

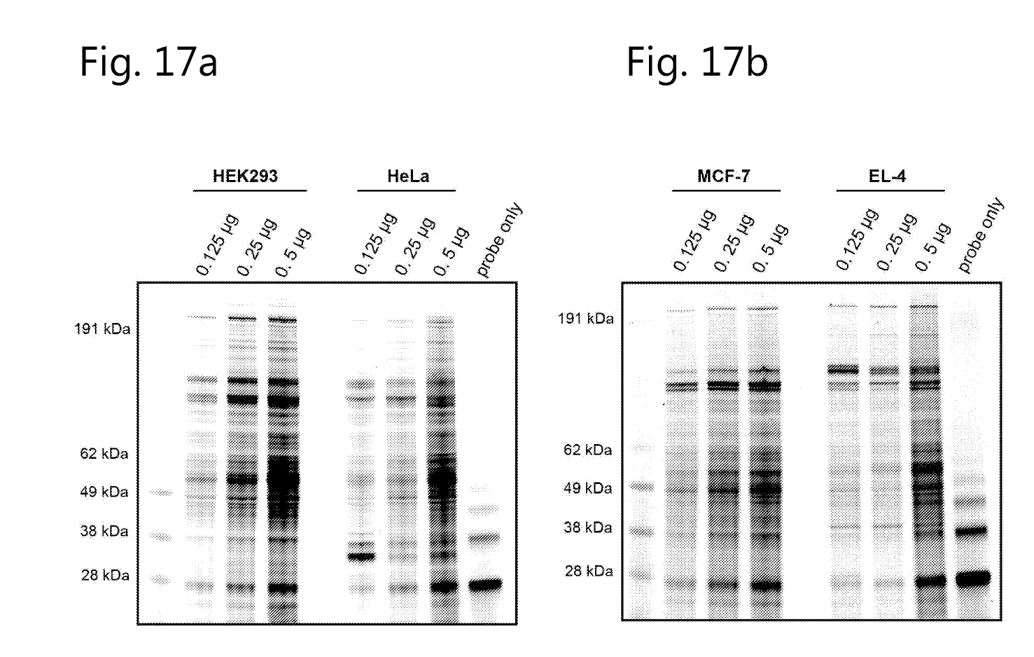

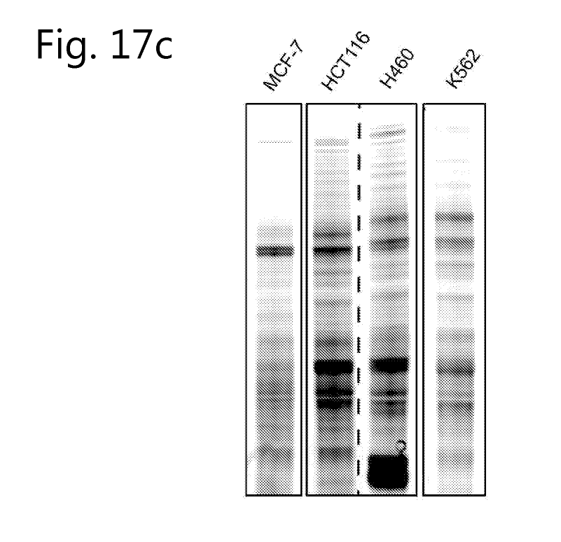

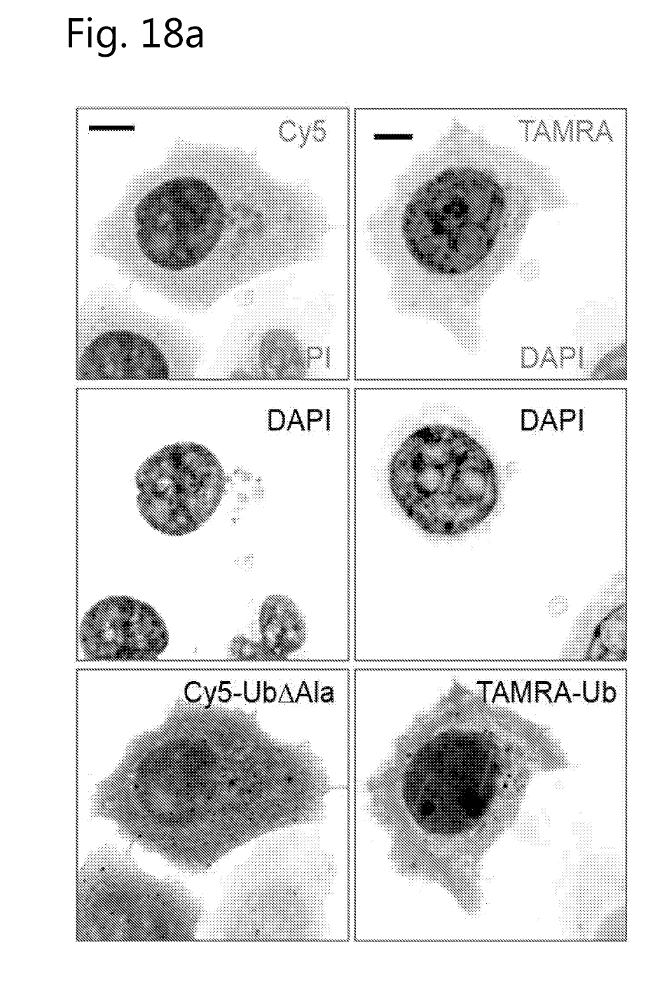

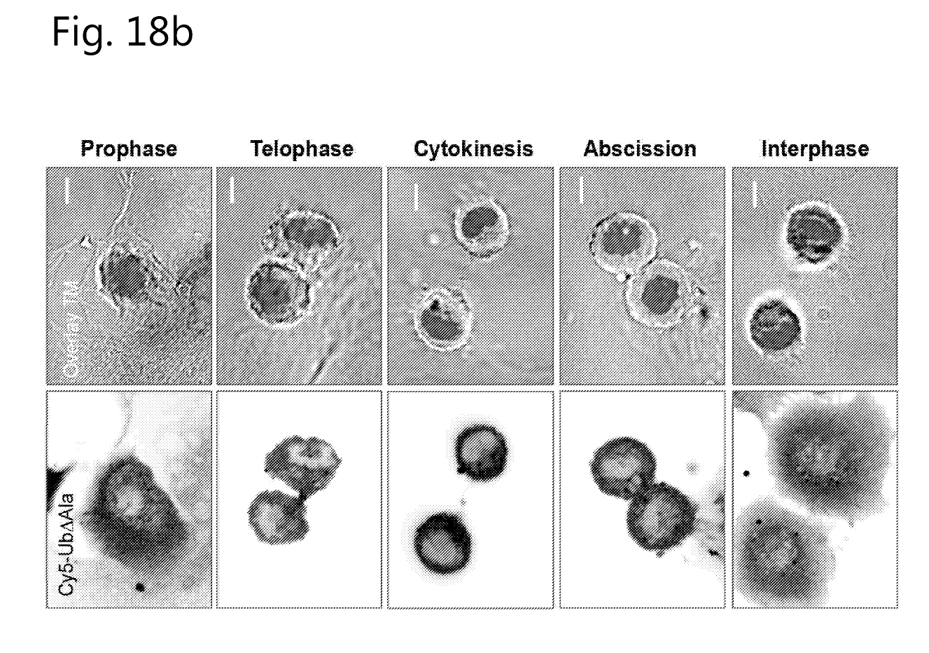

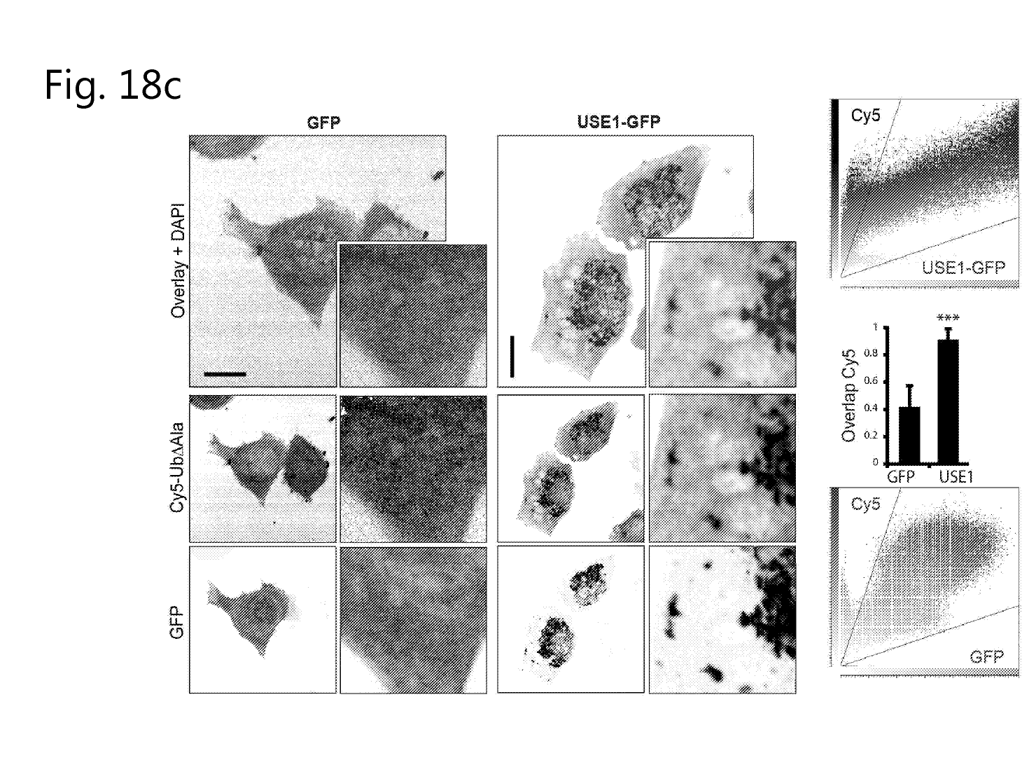

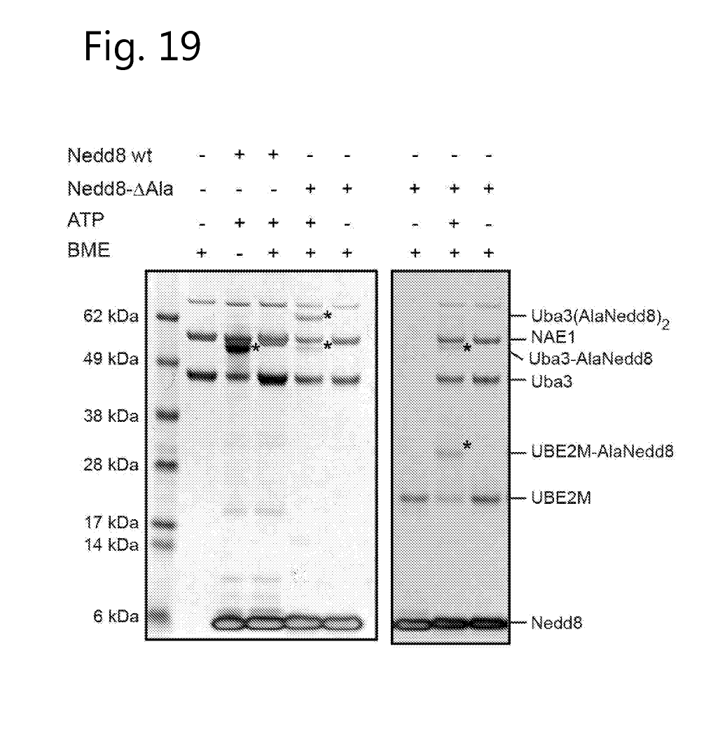

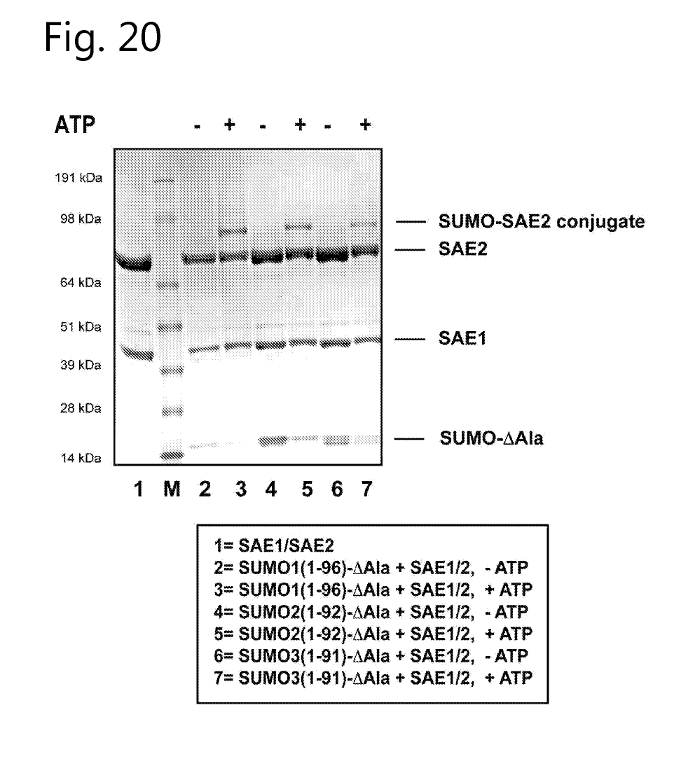

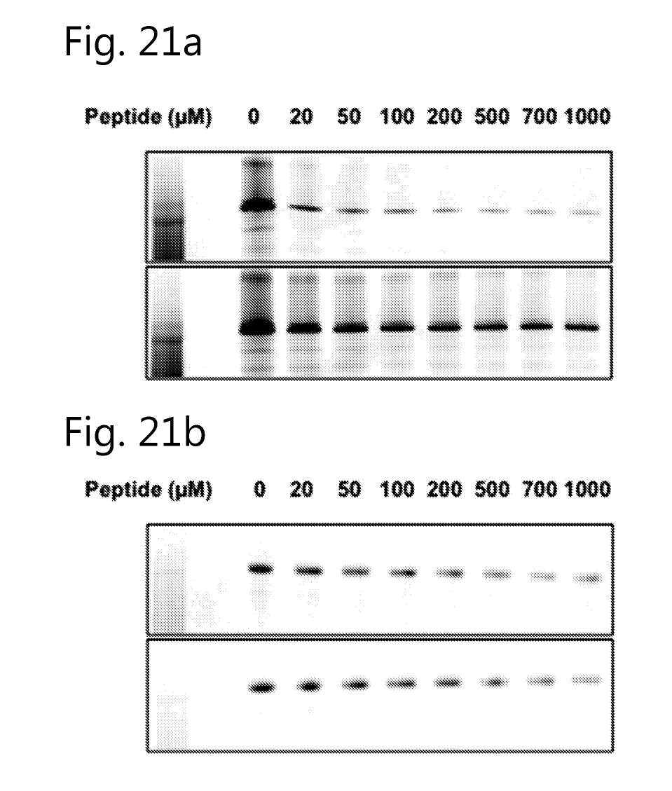

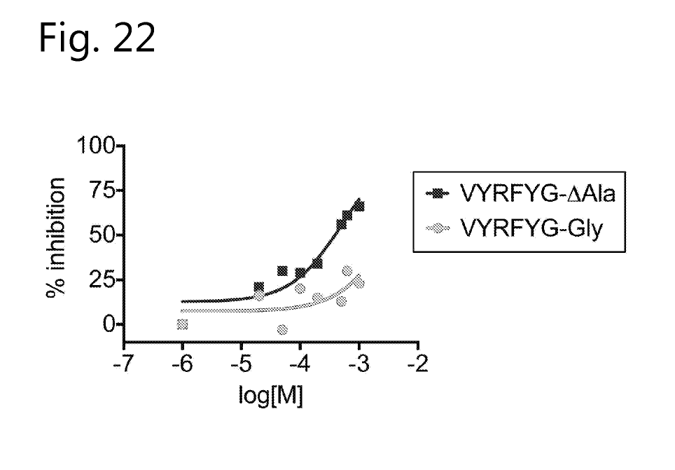

1. Adenylating enzyme (AE) substrate analogues having the structure: 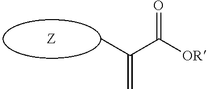
wherein R′ represents hydrogen or a moiety represented by the formula 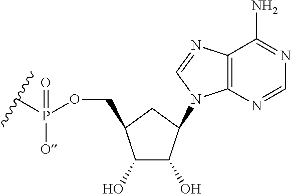
and wherein 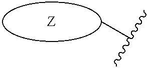
represents an organic moiety selected from the group consisting of peptides, hydrocarbons, carbohydrates and low-molecular weight organic moieties; said compound having the capability of binding to an adenylating enzyme (AE).

2. The adenylating enzyme (AE) substrate analogue according to claim 1, wherein 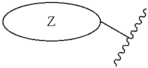
represents an organic moiety selected from the group consisting of peptides, hydrocarbons, carbohydrates and low-molecular weight organic moieties, said organic moiety being identical to or resembling the corresponding part of a C-terminal carboxylate containing AE substrate.

3. The adenylating enzyme (AE) substrate analogue according to claim 1, wherein the capability of the compound binding to an adenylating enzyme (AE) is established using a competition binding assay, wherein an IC₅₀ value of less than 10 μM is indicative of said capability of binding to an adenylating enzyme (AE).

4. An adenylating enzyme (AE) substrate analogue having the structure according to formula (I): 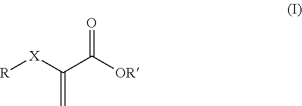
wherein R′ represents hydrogen or a moiety represented by the formula 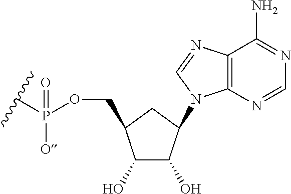
and wherein
X represents —NH— or —O—; and
R represents a peptide, or R represents
a peptide having the structure of formula (ii) 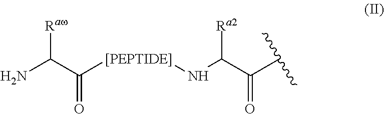
wherein R^{a#} represents an amino acid side chain identical to the amino acid side chain of the amino acid at the corresponding position in a reference AE substrate peptide; and [PEPTIDE] represents a peptide chain having the amino acid sequence —[P^{ω-1}-P³]-wherein P^{#} represents an amino acid residue identical to the amino acid residue in the corresponding position in said reference AE substrate peptide, wherein the positions are defined relative to C-terminal amino acid position, P¹ representing the C-terminal amino acid residue and P^{ω} representing the N-terminal amino acid residue;
an N-terminally truncated variant of said peptide having the structure of formula (ii), said N-terminally truncated variant comprising a number of amino acid residues of (1) equal to or higher than 1, (2) equal to or higher than 2, or (3) equal to or higher than 5; or
a homologue of said peptide or N-terminally truncated variant thereof; or
wherein
X represents —NH— or —O—; and
R represents a group having the structure of formula (iii) 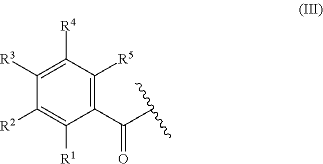
wherein R⁵ is an optionally substituted carboxylic acid, carboxylate ester or a carboxylic acid isostere;
R¹-R⁴ are independently selected from hydrogen, hydroxy, amino, halo, cyano, nitro, —CF₃, —CHF₂, —CH₂F, trifluoromethoxy, azido, (C₁-C₆)alkoxy, (C₁-C₆)alkanoyloxy, (C₁-C₆)alkyl, (C₂-C₆)alkenyl and (C₂-C₆)alkynyl, with the proviso that (1) at least 2 of R¹-R⁴ or (2) at least 3 of R¹-R⁴ represent hydrogen; or
wherein
X represents —CH₂—; and
R represents a fatty acid tail remnant, or R represents a C_{ω}-C_{γ}— aliphatic saturated or mono- or polyunsaturated carbon atom chain, identical to the C_{ω}-C_{γ}— part of an AE substrate fatty acid, wherein the carbon atoms are designated relative to the fatty acid carboxylate group, C_{α} represents the carbon atom adjacent to the carboxylate carbon atom of the fatty acid and C_{ω} represents the terminal carbon atom of the fatty acid tail.

5. The adenylating enzyme (AE) substrate analogue according to claim 4, wherein the adenylating enzyme is an E1 enzyme involved in the ubiquitin conjugation pathway, an E1 enzyme involved in the SUMO conjugation pathway, an E1 enzyme involved in the NEDD8 conjugation pathway, or an enzyme involved in the ISG15 pathway.

6. The adenylating enzyme (AE) substrate analogue according to claim 4, wherein X represents —NH— and R represents a peptide selected from the group consisting of Ub(1-75), NEDD8(1-75), ISG15(1-156), SUMO1(1-96), SUMO2(1-92), SUMO3(1-91), UFM1(1-82), FUBI(1-73), FAT10(1-164); Urm1(1-100); FAU(1-73); ATG12(1-139); ATG8(1-115) GABARAP(1-115); GABARAPL1(1-115); GABARAPL2(1-115); MAP1LC3A(1-119); MAP1LC3B(1-119); MAP1LC3B2(1-119); and MAP1LC3C(1-125) or an N-terminally truncated variant thereof; or an N-terminally truncated variant thereof or a homolgue of said peptide or an N-terminally truncated variant thereof.

7. The adenylating enzyme (AE) substrate analogue according to claim 4, selected from the group consisting of Ub(1-75)-ΔAla, NEDD8(1-75)-ΔAla, ISG15(1-156)-ΔAla, SUMO1(1-96)-ΔAla, SUMO2(1-92)-ΔAla, SUMO3(1-91)-ΔAla, Ufm1(1-82)-ΔAla, FUBI(1-73)-ΔAla, Fat10(1-164)-ΔAla, Urm1(1-100)-ΔAla, FAU(1-73)-ΔAla, ATG12(1-139)-ΔAla, GABARAP(1-115)-ΔAlaa, GABARAPL1(1-115)-ΔAla, GABARAPL2(1-115)-ΔAla, MAP1LC3A(1-119)-ΔAla, MAP1LC3B(1-119)-ΔAla, MAP1LC3B2(1-119)-ΔAla, and MAP1LC3C(1-125)-ΔAla, or a homolgue of said peptide or an N-terminally truncated variant thereof.

8. The adenylating enzyme (AE) substrate analogue according to claim 4, wherein X represents —NH₂—; and —R represents a structure according to formula (IV) 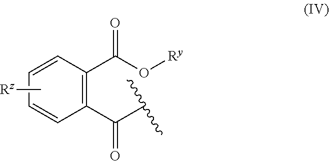
wherein
R^{Y} is selected from hydrogen and C₁-C₆ alkyl; and
R^{z} is an optional substituent selected from hydroxy, amino, halo, cyano, nitro, —CF₃, —CHF₂, —CH₂F, trifluoromethoxy, azido, (C₁-C₆)carboxy, (C₁-C₆)alkoxy, (C₁-C₆)alkanoyloxy, (C₁-C₆)alkyl, (C₂-C₆)alkenyl and (C₂-C₆)alkynyl.

9. A method for producing an adenylating enzyme (AE) substrate analogue comprising the steps of:
selecting an adenylating enzyme of interest;
selecting a natural or non-natural substrate or ligand for the adenylating enzyme comprising a C-terminal carboxylate in the moiety capable of interacting with the adenylating enzyme;
modifying the C-terminal carboxylate moiety of said adenylating enzyme substrate by introducing a methylene group at the carbon atom in the α-position relative to the C-terminal carboxylate group.

10. The method according to claim 9, comprising
selecting an adenylating enzyme of interest;
selecting a natural substrate peptide or protein for the adenylating enzyme;
carrying out a chemical and/or biological synthesis of the selected peptide substrate or protein for the adenylating enzyme, wherein said synthesis comprises substituting the C-terminal amino acid residue of the selected natural substrate peptide or protein by a dehydroalanine residue.

11. Adenylating enzyme (AE) substrate analogue obtainable by the method defined in claim 9.

12. A method selected from the group consisting of:
(a) a method for capturing an adenylating enzyme comprising capturing an adenylating enzyme in a biological matrix in vitro or ex vivo using an adenylating enzyme (AE) substrate analogue as defined in claim 1;
(b) a method of therapeutic treatment or prophylactic treatment of a subject in need of such treatment comprising treating said subject with an adenylating enzyme substrate (AE) analogue according to claim 1; and
(c) a method for treating or preventing a disease involving the action of an adenylating enzyme and/or a pathway involving an adenylating enzyme; and
(d) a diagnostic method including using an adenylating enzyme (AE) substrate analogue as defined in claim 1.

13. The method according to claim 12, wherein the method comprises (a).

14. The method according to claim 12, wherein the method comprises (b).

15. The method according to claim 12, wherein the method comprises (c).

16. The method according to claim 12, wherein the method comprises (d).

17. The adenylating enzyme (AE) substrate analogue according to claim 6, wherein the peptide is selected from the group consisting of Ub(1-75), NEDD8(1-75), ISG15(1-156), SUMO1(1-96), SUMO2(1-92), SUMO3(1-91), Ufm1(1-82), Fau(1-73) Fat10(1-164), or a homolgue of said peptide or an N-terminally truncated variant of thereof.

18. The adenylating enzyme (AE) substrate analogue according to claim 7, wherein the peptide is selected from the group consisting of Ub(1-75)-ΔAla, NEDD8(1-75)-ΔAla, ISG15(1-156)-ΔAla, SUMO1(1-96)-ΔAla, SUMO2(1-92)-ΔAla, SUMO3(1-91)-ΔAla, Ufm1(1-82)-ΔAla, Fau(1-73)-ΔAla and Fat10(1-164)-ΔAla, or a homolgue of said peptide or an N-terminally truncated variant thereof.
